(19) United States
(12) Patent Application Publication
(10) Pub. No.: US 2007/0010438 A1
(43) Pub. Date: Jan. 11, 2007

(54) Tumor treatment using beta-sheet peptides and radiotherapy

(76) Inventors: Kevin H. Mayo, Minneapolis, MN (US); Ruud P.M. Dings, St. Paul, MN (US); Robert J. Griffin, Minnetonka, MN (US)

Correspondence Address: MUETING, RAASCH & GEBHARDT, P.A., P.O. BOX 581415, MINNEAPOLIS, MN, 55458, US

(21) Appl. No.: 11/176,984

(22) Filed: Jul. 8, 2005

(51) Int. Cl. A61K 38/17; A61N 5/00

(52) U.S. Cl. 514/12; 600/1

(57) ABSTRACT

The invention relates to the use of designed β-sheet peptides together with radiation therapy for cancer treatment. The β-sheet peptides, which were designed using portions from several α-chemokines, exhibit activity as radiosensitizing agents, and have demonstrated synergism with radiation therapy for cancer treatment. The β-sheet peptides also exhibit activity as angiogenesis inhibitors.

### GOVERNMENT FUNDING

The present invention was made with government support under Grant Nos. CA 096090 and CA 044114, awarded by the National Institutes of Health. The Government may have certain rights in this invention.

### BACKGROUND

Controlling tumor growth by targeting tumor vasculature remains the subject of intense investigation. As angiogenesis is required in the growth of tumors, anti-angiogenic agents have been investigated as potential antitumor agents. The search for anti-angiogenic agents has focused on controlling two of the processes that promote angiogenesis: the growth and adhesion of endothelial cells (EC). Efforts have focused on ECs primarily because ECs are more accessible than other cells to pharmacologic agents delivered via the blood, and are genetically stable and thus not easily mutated into drug resistant variants. Most anti-angiogenic agents have been discovered by identifying endogenous molecules that inhibit EC growth. This traditional approach has produced a number of anti-angiogenic agents, such as platelet factor-4 (PF4), thrombospondin, tumor necrosis factor (TNF), interferon-γ inducible protein-10, angiostatin, endostatin, vasostatin, and bactericidal-permeability increasing (BPI) protein. In toto, about forty anti-angiogenic agents, identified using various approaches, are currently known. While a number of anti-angiogenic agents have been developed, the need for better angiogenesis inhibitors and improved methods for their use is evidenced by the absence of any major clinical breakthroughs (Fogarty, M., The Scientist 16, 33-35 (2002)) and the paucity of markers of antiangiogenic therapy that can be monitored during treatment (Kerbel, R. S., Carcinogenesis 21, 505-15 (2000)).

Angiogenesis inhibitors may be used to complement other antitumor techniques. Radiotherapy and chemotherapy are used against many new cases of cancer reported annually in the United States. Although the primary tumor is often controlled with radiotherapy and/or chemotherapy, at least temporarily, there remain over 500,000 cancer-related deaths annually in the United States alone (NCI Surveillance, Epidemiology and End results program and Centers for Disease Control and Prevention). Results of the first successful phase III clinical trial using an antiangiogenic agent (bevacizumab/Avastin) in combination with a chemotherapeutic regimen were recently announced (Yang et al., N. Engl. J. Med. 349, 427-34 (2003); listed in Clin. Colorectal Cancer 3, 85-88 (2003)). This trial demonstrates that angiogenesis inhibitors, when used in combination with conventional chemotherapy, appear to be a powerful tool to combat cancer in patients.

In animal models, combining angiogenesis inhibitors with radiation therapy (Mauceri et al., Nature 394, 287-91 (1998); Griffin et al., Cancer Res. 62, 1702-06 (2002)), gene therapy (Wilczynska et al., Acta Biochim. Pol. 48, 1077-84 (2001)), or chemotherapy (Teicher et al., Cancer Res 52, 6702-4 (1992); Teicher et al., Eur. J. Cancer 32A, 2461-6 (1996); Herbst et al., Cancer Chemother. Pharmacol. 41, 497-504 (1998)) has been shown to be potentially beneficial. In the case of radiation therapy, apoptosis of endothelial cells has been recognized as a critical component of the radiation response (Garcia-Barros et al., Science 300, 1155-59 (2003); Folkman et al., Science 293(5528), 227-8 (2001)), independent of tumor oxygenation during radiation (Lee et al., Cancer Res. 60, 5565-70 (2000)). The list of antiangiogenic agents demonstrated to enhance the antitumor effects from radiation therapy includes, for example, thrombospondin-1 (Rofstad et al., Cancer Res. 63, 4055-61 (2003)), angiostatin (Gorski et al., Cancer Res. 58, 5686-9 (1998)), various receptor tyrosine kinase inhibitors (Griffin et al., Cancer Res. 62, 1702-06 (2002), and anti-VEGF and VEGFR antibodies (Gorski et al., Cancer Res. 59, 3374-8 (1999)). However, many of these agents are difficult and expensive to produce and/or have documented toxicity issues. Thus, other ways or methods of treating tumors is needed.

### SUMMARY OF THE INVENTION

The present invention demonstrates that infusion of β-sheet peptides in combination with radiation results in tumor growth inhibition. Furthermore, injection of β-sheet peptides before, after, or during radiation treatment preferably sensitizes endothelial cells to radiation and significantly prolongs radiation-induced tumor growth delay. Changes in tumor histology and in vitro tissue culture studies strongly suggest that combination treatment with β-sheet peptides and radiation is an effective tumor treatment strategy.

Accordingly, in one aspect, the present invention provides a method of treating a patient with a tumor that includes delivering radiation to the patient and administering to the patient a β-sheet peptide. The β-sheet peptide is a water soluble peptide having at least 35% to 55% amino acids having hydrophobic side chains in which the ratio of amino acids having positively charged side chains amino acids to amino acids having negatively charged side chains is at least 2:1, and in which at least two of the amino acids having hydrophobic side chains are positioned in the peptide chain with an intervening turn sequence in a manner such that the two amino acids having hydrophobic side chains are capable of aligning in a pairwise fashion to form a β-sheet structure. Furthermore, the β-sheet peptide is water soluble under physiological conditions, and the peptide forms β-sheet structures.

In further aspects of the method of the invention, the turn sequence of the β-sheet peptide is LXXGR(SEQ ID NO:33) and X is independently selected from the group consisting of K, N, S, and D, and/or the β-sheet peptide consists of 28 to 33 amino acids.

In a preferred aspect of the invention, the radiation and the β-sheet peptide treat the patient with a tumor synergistically. In one aspect, the β-sheet peptide of the method is administered before delivering radiation to the patient and the β-sheet peptide radiosensitizes the tumor to radiation. One type of cells that may be radiosensitized is endothelial cells. In a further aspect, the β-sheet peptide is administered within 24 hours of delivering radiation to the patient.

The invention may include a variety of additional aspects. For instance, the β-sheet peptide used in the method of the invention is administered along with a pharmaceutically acceptable carrier. The method may also include inhibition of angiogenesis by the β-sheet peptide. Radiation used in the method may include gamma ray or x-ray radiation.

The method of the invention may be used to treat a variety of tumors. In one aspect, the tumor is a solid tumor. In a further aspect, the solid tumor is selected from the group consisting of carcinomas, sarcomas, and lymphomas. For solid tumors, the radiation is preferably a daily dose of about 50 to 70 grays. In a further aspect, the solid tumor is present in the brain, breast, cervix, larynx, lung, pancreas, prostate, skin, spine, stomach, or uterus. The tumor treated by the method of the invention may also be a leukemia. For leukemia, the radiation is preferably a daily dose of about 20 to 40 grays.

In an additional aspect of the method of the invention, the β-sheet peptide is selected from the group consisting of βpep-1 through βpep-30 (SEQ ID NOS:1-30) and their derivatives. In a further aspect, the β-sheet peptide is βpep-25 and its derivatives. In yet a further aspect, the β-sheet peptide is βpep-25.

The invention also provides a method of treating a patient with a tumor that includes delivering gamma or x-ray radiation to the patient and administering to the patient βpep-25 in a pharmaceutically acceptable carrier. In one aspect of this method, the radiation and βpep-25 treat the patient with a tumor synergistically. In a further aspect, the radiation and βpep-25 provide a synergistic effect of 200% or more.

“Amino acid” is used herein to refer to a chemical compound with the general formula: NH₂—CRH—COOH, where R, the side chain, is H or an organic group. Where R is organic, R can vary and is either polar or nonpolar (i.e., hydrophobic). The amino acids of this invention can be naturally occurring or synthetic (often referred to as nonproteinogenic). As used herein, an organic group is a hydrocarbon group that is classified as an aliphatic group, a cyclic group or combination of aliphatic and cyclic groups. The term “aliphatic group” means a saturated or unsaturated linear or branched hydrocarbon group. This term is used to encompass alkyl, alkenyl, and alkynyl groups, for example. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” refers to mono- or polycyclic aromatic hydrocarbon groups. As used herein, an organic group can be substituted or unsubstituted. One letter and three letter symbols are used herein to designate the naturally occurring amino acids. Such designations including R or Arg, for Arginine, K or Lys, for Lysine, G or Gly, for Glycine, and X for an undetermined amino acid, and the like, are well known to those skilled in the art.

The terms “polypeptide” and “peptide” as used herein, are used interchangeably and refer to a polymer of amino acids. These terms do not connote a specific length of a polymer of amino acids. Thus, for example, the terms oligopeptide, protein, and enzyme are included within the definition of polypeptide or peptide, whether produced using recombinant techniques, chemical or enzymatic synthesis, or naturally occurring. This term also includes polypeptides that have been modified or derivatized, such as by glycosylation, acetylation, phosphorylation, and the like.

The term “water-soluble” is used herein to refer to compounds, molecules, and the like, including the peptides of this invention, that are preferably readily dissolved in water. The compounds of this invention are readily dissolved in water if about 1 mg of the compound dissolves in 1 ml of water having a temperature of about 35-45° C. More preferably, the peptides of this invention will have a water solubility of at least about 10 mg/ml and often of at least about 20 mg/ml. Even more preferably, the peptides are soluble at these concentrations under physiological conditions, including a pH of about 7.0-7.4 and a salt concentration of about 150 mM NaCl.

The term “hydrophobic amino acid side chain” or “nonpolar amino acid side chain,” is used herein to refer to amino acid side chains having properties similar to oil or wax in that they repel water. In water, these amino acid side chains interact with one another to generate a nonaqueous environment. Examples of amino acids with hydrophobic side chains include, but are not limited to, valine, leucine, isoleucine, phenylalanine, and tyrosine.

The term “polar amino acid side chain” is used herein to refer to groups that attract water or are readily soluble in water or form hydrogen bonds in water. Examples of polar amino acid side chains include hydroxyl, amine, guanidinium, amide, and carboxylate groups. Polar amino acid side chains can be charged or non-charged.

The term “non-charged polar amino acid side chain” or “neutral polar amino acid side chain” is used herein to refer to amino acid side chains that are not ionizable or do not carry an overall positive or negative charge. Examples of amino acids with non-charged polar or neutral polar side chains include serine, threonine, glutamine, and the like.

The term “positively charged amino acid side chain” refers to amino acid side chains that are able to carry a full or positive charge and the term “negatively charged amino acid side chain” refers to amino acid side chains that are able to carry a negative charge. Examples of amino acids with positively charged side chains include arginine, histadine, lysine, and the like. Examples of amino acids with negatively charged side chains include aspartic acid and glutamic acid, and the like.

The term “self-association” refers to the spontaneous association of two or more individual peptide chains or molecules irrespective of whether or not the peptide chains are identical.

The term “tumor” refers a collection of cells that have developed cancer. The collection of cells may form an aggregate, as in solid tumors, or may be diffuse, as in leukemias. Cancer cells contain genetic damage that has resulted in the relatively unrestrained growth of the cells. The genetic damage present in a cancer cell is maintained as a heritable trait in subsequent generations of the cancer cell line. The genetic damage found in cancer cells is generally found in oncogenes, and tumor suppressor genes, but can also occur in genes governing immunity, cell motility, or angiogenesis.

Unless otherwise specified, “a,” “an,” “the,” and “at least one” are used interchangeably and mean one or more than one.

### BRIEF DESCRIPTION OF THE FIGURES

FIG. 1 schematically illustrates the alignment of β-sheet regions from the polypeptides PF4, IL-8 and GRO polypeptides. β-sheet residues are blocked-in and lines connect the residues that are paired in the chain. The C-termini in the sequences were synthesized in the amide form. Numbering shown below the sequence is that from native PF4.

FIG. 2 provides graphs showing the mean tumor growth curves for the human ovarian carcinoma (MA148) and the murine syngeneic mammary carcinoma (SCK) in mice treated with βpep-25, radiation or combination treatment. FIG. 2A shows the mean tumor growth curve of the MA148 xenograft. Groups shown are defined as follows: —▪—, vehicle containing BSA (n=13); —●—, βpep-25 (10 mg/kg/day for 28 days, n=10); —▴—, radiation (n=10); —▾—, combination group (n=10). FIG. 2b shows the mean tumor growth curve of SCK tumors. Groups shown are defined as follows: —▪—, vehicle containing BSA (n=12); —●—, βpep-25 (20 mg/kg/day for 14 days, n=12); —▴—, radiation (n=13); —▾—, a combination group (n=13). Radiation was administered locally at the time points and amount indicated by arrows. Data shown are means of tumor volume. Error bars, SEs. In FIG. 2B, the error bars are shown until the first animal death in each group, the means thereafter represent the average tumor volumes of the mice still present.

FIG. 3 is a picture showing the results of histochemical analysis. Double staining of tumor cross-sections that were stained for vessel density, apoptosis and proliferation. Microvessel density (MVD) is revealed by PE-labeled anti-CD31 antibody staining, apoptosis staining is revealed by using the TUNEL assay, and proliferation is revealed by PE-labeled anti-PCNA, all as indicated. FIG. 3A shows MA148 tumor section staining. Specimens of the combination treatment were not available for histology due to tumor regression during course of treatment. FIG. 3B shows SCK tumor section staining. Quantifications are given in Table 2 and the arrows indicate double staining. Original magnification 200×.

FIG. 4 provides graphs showing βpep-25 affects tumor physiology. FIG. 4A shows SCK tumor blood perfusion is reduced by 5 days of i.p. injection of 10 mg/kg βpep-25 treatment measured by ⁸⁶Rb uptake. FIG. 4B shows that the median partial pressure of O₂ (pO₂) was significantly (p<0.05) reduced in SCK tumors by 7 daily i.p. injections of 20 mg/kg βpep-25 treatment.

FIG. 5 provides graphs showing the relative effectiveness of βpep-25 and angiostatin as radiosensitizers. FIG. 5A shows i.p. injections of βpep-25 (20 mg/kg) were given on days −1, 0, and 1 to SCK bearing mice. Radiation (10 Gy) was applied on days 0 and 1, 2 hours after βpep-25 injection. FIG. 5B shows i.p. injections of angiostatin (25 or 50 mg/kg) were given on days −1, 0, and 1 in the SCK tumor mouse model. Radiation (10 Gy) was applied on days 0 and 1, 2 hours after injection of angiostatin. Data shown are means of tumor volume. Error bars (SEs) are shown until the first animal death in each group, the means thereafter represent the tumor volumes of the mice still alive.

FIG. 6 is a bar graph showing the relative changes in the volume of SCCVII tumors after injection of 10 mg/kg of βpep-25 or exposure to 8 Gy alone or combined. Tumor-bearing animals were given i.p. injection of 10 mg/kg βpep-25 on days 1 and 3 after irradiation. Each data point is average tumor volume±1 SE measured in 7-10 animals per treatment group.

FIG. 7 is a bar graph showing typical hematoxylin and eosin (H&E) or pimonidazole staining of SCCVII tumors at 5 days after first injection 10 mg/kg of βpep-25 (twice injections) and/or radiation exposure of 8Gy, 40× and 400× magnification. At least three animals per group were analysed. Images shown are representatives of each treatment group.

FIG. 8 is a graph showing the frequency distribution of measured intra-tumor pO2 in saline-treated, βpep-25 alone, 8 Gy alone and 8 Gy and βpep-25 combined, constructed as function of oxygen tension, with grouping in 2 mmHg intervals.

FIGS. 9A-9H provide power doppler images of tumor bearing mice. Power doppler tumor images of vehicle (saline) treated mouse on day 3 and day 7 are shown in 9A and 9B, respectively. Power doppler tumor images of a βpep-25 treated mouse on day 3 and 7 are shown in FIGS. 9C and 9D, respectively. Power doppler tumor images of an 8 Gy treated mouse are shown on day 7 and day 14 in 9E and 9F, respectively. Power doppler tumor images of a combination (βpep-25 and 8 Gy) treated mouse on day 7 and day 14 are shown in 9G and 9H, respectively. Images shown are representative of the mean.

FIG. 10 provides graphs showing βpep-25 specifically targets endothelial cells and enhances the anti-proliferative activity of radiation. FIG. 10A shows βpep-25 alone specifically inhibits endothelial cell proliferation and does not affect MA148 and SCK tumor cells. FIG. 10B shows dose response curve of 72 hours of βpep-25 exposure combined with radiation exposure 4 hours after the start of βpep-25 treatment showing an enhanced effect on proliferating HUVEC. FIG. 10C shows βpep-25 radiosensitizes endothelial cells. FIG. 10D shows clonogenicity of MEC is reduced by 4 hours of βpep-25 exposure or 2.5 Gy and combining βpep-25 with radiation caused a significant decrease (p less than 0.05) in clonogenicity compared to either treatment alone. βpep-25 alone for 4 hours has little to no effect on colony formation of MA148 and SCK cells, as shown in FIGS. 10E and 10F, respectively. The survival was reduced by 40-50% by exposure to 5 Gy alone, but not further decreased when βpep-25 was combined with radiation.

FIG. 11 graphically illustrates ³H-Thymidine incorporation data for two different types of endothelial cells with peptide (βpep-1 through βpep-24) concentrations of 2×10⁻⁶ M. FIG. 11A provides ³H-Thymidine incorporation data for FBHEC cells and FIG. 11B provides ³H-Thymidine incorporation data for HUVEC cells.

### DETAILED DESCRIPTION OF ILLUSTRATIVE EMBODIMENTS OF THE INVENTION

The present invention provides methods of treating tumors using a combination of radiation and designed water-soluble β-sheet forming peptides. In one aspect of the invention, the β-sheet peptides act as radiosensitizers to increase tumor susceptibility to treatment by radiation. In a further aspect, the β-sheet peptides interact with radiation therapy to provide synergistic effects in treating tumors.

The β-sheet peptides of the present invention were designed, in part, using portions of chemokines. Chemokines are small, chemotactic cytokines that direct migration of leukocytes, activate inflammatory responses, participate in many other pleiotropic functions, including regulation of tumor growth, and have been proposed for use in anticancer therapies (Frederick et al., Exp. Rev. in Mol. Med., 1-17,(2001)). Chemokines have also been used as a starting point to design water-soluble β-sheet forming peptides, as described in U.S. Pat. No. 6,486,125. These β-sheet peptides contain an appropriate percent composition of amino acids with hydrophobic side chains and proper placement in the amino acid sequence to promote self-association-induced structural collapse and stability, providing them with a β-sheet structure and water solubility. Many of these peptides have been shown to possess pharmaceutical activity, including endotoxin neutralizing activity, antibacterial activity, and anti-angiogenic activity, as described in U.S. Patent Publication No. 20030153502. One of these peptides, βpep-25, also referred to by the trade name ANGINEX, is a β-sheet forming peptide 33mer with potent anti-angiogenesis activity both in vitro (Griffioen et al., Biochem J. 354, 233-42 (2001)) and in vivo (Dings et al., Cancer Lett.;194, 55-66 (2003); van der Schaft et al., Faseb J. 16, 1991-93 (2002)). βpep-25 is water soluble, shelf-stable, and non-toxic in animals. In combination with a sub-optimal dose of the chemotherapeutic drug carboplatin, βpep-25 treatment provided a synergistic effect and led to an improved outcome compared to either treatment alone. Dings et al., Cancer Res. 63, 382-85 (2003).

Structure and Preparation of Designed Water-Soluble β-Sheet Forming Peptides

Water-soluble β-sheet forming peptides have been designed as described in U.S. Pat. No. 6,486,125, issued to Mayo et al. These peptides were designed to form soluble folded peptides that avoided the problems of precipitation or over-solvation caused by forming a peptide that is either not soluble enough or with too high an affinity for water to form β-sheets. β-sheets, also referred to as β-pleated sheets, are a periodic structural motif found in many proteins, and are categorized as a form of protein secondary structure. The polypeptide chain forming a β-sheet includes long stretches in which the polypeptide chain is almost fully extended, and in which adjacent chains can run in the same or opposite direction, forming parallel and antiparallel β-sheets, respectively. The axial distance between adjacent amino acids is about 3.5 Å, and the sheet is stabilized primarily by hydrogen bonds that form between amine and carbonyl groups in adjacent polypeptide chains. The adjacent polypeptides forming the sheet, also referred to as strands, are typically connected by β-turns, in which a short peptide (e.g., a tetrapeptide) forms a hairpin turn, again stabilized by hydrogen bonding between carbonyl and amine groups. The amino acids making up the β-turn are referred to herein as a turn sequence.

The β-sheet peptides used in the present invention were designed taking into account a variety of parameters. These include, for example, the number or percentage composition of amino acids with positively and negatively charged side chains, the number or percentage composition of amino acids with non-charged polar side chains, the number or percentage composition of amino acids with hydrophobic side chains, proper placement and pairing of amino acids in the sequence and in space, and specific turn character. The specific turn character refers to the composition of side chains of the amino acids positioned in the turn sequence.

When the number of amino acids with positively and negatively charged side chains is about equal, intermolecular electrostatic interactions shift the solvation-precipitation equilibrium to the precipitate state. Adjusting the overall net charge of the peptide to have mostly amino acids with positively charged side chains greatly improves solubility. Inter-peptide charge repulsion may also help to reduce precipitation. In a preferred embodiment of the invention, the ratio of amino acids with positively charged side chains to amino acids with negatively charged side chains in a β-sheet peptide is at least 2:1. Preferably the ratio of amino acids with positively charged side chains to amino acids with negatively charged side chains is no greater than 3:1; however, this invention also considers larger ratios of amino acids with positively charged side chains to amino acids with negatively charged side chains including, but not limited to, 4:1, 5:1, 6:1 or greater.

While the β-sheet peptides are preferably water-soluble, they must not be too soluble or they may become over-solvated. When the number of amino acids with polar side chains is too high and other stabilizing forces are too low, desired protein folding may be hindered by intermolecular peptide-water associations. Therefore, a high content of amino acids with short chain polar side chains such as serine and threonine (the hydroxylated amino acids) is not preferred. The peptides of the present invention preferably contain less than 100%, preferably less than 50%, more preferably, less than 20% amino acids with non-charged polar side chains.

An appropriate percent composition of amino acids with hydrophobic side chains and proper placement in the sequence of such amino acids promotes self-association-induced folding and stability. The trade-off is to adjust the percent composition of amino acids with hydrophobic side chains to avoid insolubility, while promoting folding and structure formation. The β-sheet peptides thus preferably contain 35% to 55% amino acids with hydrophobic side chains, and in particularly preferred embodiments, 40% to 50% amino acids with hydrophobic side chains. In preferred embodiments of this invention, the hydrophobic amino acids, or combination thereof, are aliphatic, although aromatic hydrophobic amino acids may be used. Percentages are reported as the number of specified amino acids relative to the total number of amino acids in the peptide chain.

To generate a compact fold in a β-sheet peptide, side-chain pairing and packing should be optimized by encouraging desired hydrophobic interactions. Choosing the proper placement of amino acids with hydrophobic side chains in the sequence and combination of hydrophobic side-chain triplets across the strands as well as between strands in the self-associated peptide is an important aspect of designing stable β-sheet folds. Preferably, the amino acids are also positioned in the folded peptide to form a substantially hydrophobic surface. More preferably, the amino acids are positioned in the folded peptide such that one peptide molecule is capable of self-associating with another peptide molecule to form a multimer.

Efficient hydrophobic side-chain packing of one sheet on top of another appears to be important for optimum folding stability and compactness. Choosing the proper placement of side chains, particularly hydrophobic side chains, in the amino acid sequence is thus important to control fold stability. Compact β-sheet folding is typically dependent on well-packed inter-strand side chain pairings. Preferably, the β-sheet peptide has at least two amino acids with hydrophobic side chains, and more preferably, three amino acids with hydrophobic side chains that are positioned to align in space to form a β-sheet structure. Between these amino acids are turn sequences to allow for these side chain pairings.

Specific turn character may be used in the β-sheet peptides to promote or stabilize a desired fold. A variety of turn sequences are known in the art. A specific novel folding initiation turn/loop sequence, KXXGR (Ilyina et al., Biochemistry 33, 13436 (1994) was used in SEQ ID NOS:1-4 (βpep-5, βpep-8, βpep-11 and βpep-1). In this sequence, each X is independently selected from the group consisting of K, N, S, and D. This sequence was positioned between two amino acids with hydrophobic side chains such that the two amino acids having hydrophobic side chains were capable of aligning in a pairwise fashion to form a β-sheet structure.

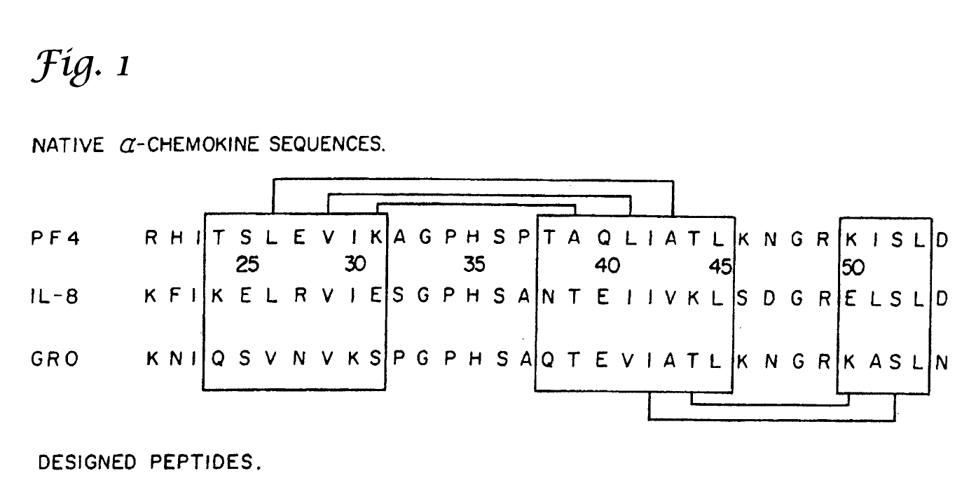

A β-sheet peptide of the invention preferably has at least 20 amino acids. Preferably the β-sheet peptides of this invention are no greater than 50 amino acids in length, and more preferably about 28 to about 33 amino acids in length. U.S. Pat. No. 6,486,152 describes how 30 particular β-sheet peptides—βpep-1 through βpep-30—were designed de novo. The peptides were prepared, in part, by using various portions of α-chemokines (e.g., platelet factor 4, interleukin-8, growth-related polypeptide (Gro-α), and neutrophil activating peptide-2), which are chemokines known to attract neutrophils. Portions of these chemokines that were used to prepare β-sheet peptides are shown in FIG. 1. As the β-sheet peptides of the present invention include significant portions of α-chemokines, they may also be referred to as α-chemokine hybrid peptides. A number of β-sheet peptides are shown in Table 1 below. All of the peptides shown in Table 1 are 33 amino acid residues long. As can be seen, the 30 amino acid sequences contain many similarities to one another. All of these β-sheet peptides are water soluble at least up to 30 mg/mL (9 mM) at pH values between pH=2 and pH=10, and all have been shown by circular dichroism (CD) and nuclear magnetic resonance (NMR) to form β-sheets and significant populations of self-association-induced β-sheet structure in water at near-physiological conditions.

TABLE 1
Amino Acid Seciuence of β-Peptides

βpep-5 (SEQ ID NO:1)
KFIVTLRVIKAGPHSPTAQIIVELKNGRKLSLD
βpep-8 (SEQ ID NO:2)
ANIKLSVEMKLFKRHLKWKIIVKLNDGRELSLD
βpep-11 (SEQ ID NO:3)
ANIKLSVEMKLFCY^{D}WKVCKIIVKLNDGRELSLD
βpep-1 (SEQ ID NO:4)
SIQDLNVSMKLFRKQAKWKIIVKLNDGRELSLD
βpep-2 (SEQ ID NO:5)
ANIKLSVKWKAQKRFLKMSINVDLSDGRELSLD
βpep-3 (SEQ ID NO:6)
HIKELQVKWKAQKRFLKMSIIVKLNDGRELSLD
βpep-4 (SEQ ID NO:7)
SIQDLNVSMKLFRKQAKWKINVKLNDGRELSLD
βpep-6 (SEQ ID NO:8)
HIKELQVRWRAQKRFLRMSIIVKLNDGRELSLD
βpep-7 (SEQ ID NO:9)
HIKELQVKMKAQKRFLKWSIIVKLNDGRELSLD
βpep-9 (SEQ ID NO:10)
ANIKLSVKWKAQKRFLKMSIIVKLNDGRELSLD
βpep-10 (SEQ ID NO:11)
ANIKLSVEMKLFCRHLKCKIIVKLNDGRELSLD
βpep-12 (SEQ ID NO:12)
ANIKLSVEMKFFKRHLKWKIIVKLNDGRELSLD
βpep-13 (SEQ ID NO:13)
ANIKLSVEFKLFKRHLKWKIIFKLNDGREFSLD
βpep-14 (SEQ ID NO:14)
SIQDLNVSMKLFRKQAKWKLIVKLNDGRELSLD
βpep-15 (SEQ ID NO:15)
SIQDLNVSMKLFRKQAKWKIILKLNDGRELSLD
βpep-16 (SEQ ID NO:16)
SIQDLNVSMKLFRKQAKWKIIAKLNDGRELSLD
βpep-17 (SEQ ID NO:17)
SIQDLNVSMKLFRKQAKWKILVKLNDGRELSLD
βpep-18 (SEQ ID NO:18)
SIQDLKVSMKLFRKQAKWKIIVKLNDGRELSLD
βpep-19 (SEQ ID NO:19)
SIQKLNVSMKLFRKQAKWKIIVKLNDGRELSLD
βpep-20 (SEQ ID NO:20)
SIQDLNVSMXLFRKQAKWKIIVKLNDGRELSLD
“X” in this sequence refers to the non-common
aminoacid norleucine
βpep-21 (SEQ ID NO:21)
SIQDLNVSLKLFRKQAKWKIIVKLNDGRELSLD
βpep-22 (SEQ ID NO:22)
SIQDLNLSMKLFRKQAKWKIIVKLNDGRELSLD
βpep-23 (SEQ ID NO:23)
SIQDLKVSLNLFRKQAKWKIIVKLNDGRELSLD
βpep-24 (SEQ ID NO:24)
SIQFLKVSLNLDRKQAKWKIIVKLNDGRELSLD
βpep-25 (SEQ ID NO:25)
ANIKLSVQMKLFKRHLKWKIIVKLNDGRELSLD
βpep-26 (SEQ ID NO:26)
SIQDLNVSMKLFRKQAKWKIIIKLNDGRELSLD
βpep-27 (SEQ ID NO:27)
SIQDLNVSMKLFRKQAKWKAIVKLNDGRELSLD
βpep-28 (SEQ ID NO:28)
SIQDLNVSMKLFRKQAKWKVIVKLNDGRELSLD
βpep-29 (SEQ ID NO:29)
SIQDLNVSMKLFRKQAKWKLILKLNDGRELSLD
βpep-30 (SEQ ID NO:30)
SIQDLNVSMKLFRKQAKWKVIIKLNDGRELSLD

The β-sheet peptides can be further modified in a variety of ways to form derivatives. These modifications include addition of organic groups to form modified polypeptides, or addition, substitution or deletion of amino acids. These modifications preferably do not eliminate or substantially reduce the biological activity of the peptide. The biological activity of a polypeptide can be determined, for example, as described in the Examples section. Conservative amino acid substitutions typically can be made without affecting biological activity.

Substitutes for an amino acid in the polypeptides of the invention are preferably conservative substitutions, which are selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can generally be substituted for another amino acid without substantially altering the structure of a polypeptide. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine, and histidine. The negatively charged (acidic) amino acids include aspartic acid and glutamic acid. Examples of preferred conservative substitutions include Lys for Arg and vice versa to maintain a positive charge; Glu for Asp and vice versa to maintain a negative charge; Ser for Thr so that a free —OH is maintained; and Gin for Asn to maintain a free NH₂.

Other amino acids and derivatives thereof that can be used include 3-hydroxyproline, 4-hydroxyproline, homocysteine, 2-aminoadipic acid, 2-aminopimelic acid, γ-carboxyglutamic acid, β-carboxyaspartic acid, ornithine, homoarginine, N-methyl lysine, dimethyl lysine, trimethyl lysine, 2,3-diaminopropionic acid, 2,4-diaminobutyric acid, homoarginine, sarcosine, hydroxylysine, substituted phenylalanines, norleucine, norvaline, 2-aminooctanoic acid, 2-aminoheptanoic acid, statine, β-valine, naphthylalanines, substituted phenylalanines, tetrahydroisoquinoline-3-carboxylic acid, and halogenated tyrosines.

Polypeptide derivatives, as that term is used herein, also include modified polypeptides. Modifications of polypeptides of the invention include chemical and/or enzymatic derivatizations at one or more constituent amino acid, including side chain modifications, backbone modifications, and N- and C-terminal modifications including acetylation, hydroxylation, methylation, amidation, and the attachment of carbohydrate or lipid moieties, cofactors, and the like.

Synthetic methods may be used to produce β-sheet peptides, as is described in U.S. Pat. No. 6,486,125. Such methods are known and have been reported (Merrifield, Science, 85, 2149 (1963), Olson et al., Peptides, 9, 301, 307 (1988)). The solid phase peptide synthetic method is an established and widely used method which is described, for example, in the following references: Stewart et al., Solid Phase Peptide Synthesis, W. H. Freeman Co., San Francisco (1969); Merrifield, J. Am. Chem. Soc., 85 2149 (1963); Meienhofer in “Hormonal Proteins and Peptides,” ed.; C. H. Li, Vol. 2 (Academic Press, 1973), pp. 48-267; Bavaay and Merrifield, “The Peptides,” eds. E. Gross and F. Meienhofer, Vol. 2 (Academic Press, 1980) pp. 3-285; and Clark-Lewis et al., Meth. Enzymol., 287, 233 (1997). Peptides can be readily purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography, and the like. Peptides can also be readily purified through binding of a fusion polypeptide to separation media, followed by cleavage of the fusion polypeptide to release a purified polypeptide.

The β-sheet peptides may also be prepared via recombinant techniques well known to those skilled in the art. A polynucleotide sequence coding for a β-sheet peptide can be constructed by techniques well known in the art. It will further be understood by those skilled in the art that owing to the degeneracy of the genetic code, a sizeable yet definite number of DNA sequences can be constructed to encode peptides having an amino acid sequence corresponding to a particular β-sheet peptide. Once the DNA sequence has been determined, it can be readily synthesized using commercially available DNA synthesis technology. The DNA sequence can then be inserted into any one of many appropriate and commercially available DNA expression vectors through the use of appropriate restriction endonucleases. A variety of expression vectors useful for transforming prokaryotic and eukaryotic cells are well known in the art. The DNA sequences coding for the peptide are inserted in frame and operably linked to transcriptional and translational control regions, such as promoters, which are present in the vector and are functional in the host cell. The DNA sequence coding for the peptide can also be inserted into a system that results in the expression of a fusion protein that contains the β-sheet peptide. For example, U.S. Pat. No. 5,595,887 describes methods of forming a variety of relatively small peptides through expression of a recombinant gene construct coding for a fusion protein that includes a binding protein and one or more copies of the desired target peptide. After expression, the fusion protein is isolated and cleaved using chemical and/or enzymatic methods to produce the desired target peptide.

Cancer Formation and Types

The β-sheet peptides of the invention can be administered to a patient (e.g., a mammal such as a human) in conjunction with radiation therapy as a method of treating cancer. Cancer is a disease of abnormal and excessive cell proliferation. Cancer generally is initiated by an environmental insult or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cancer results in an increased number of cancer cells in a patient. These cells may form an abnormal mass of cells called a tumor, the cells of which are referred to as tumor cells. The overall amount of tumor cells in the body of a patient is referred to as the tumor load. Tumors can be either benign or malignant. A benign tumor contains cells that are proliferating but remain at a specific site. The cells of a malignant tumor, on the other hand, can invade and destroy nearby tissue and spread to other parts of the body through a process referred to as metastasis.

While cancer is defined by its nature, cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system. When a tumor does not contain cysts or liquid areas, it is generally referred to as a solid tumor. Carcinomas, sarcomas, and lymphomas often form a solid tumor, whereas leukemias generally do not.

Radiation Therapy

Radiation therapy is used in conjunction with the β-sheet peptides of the invention as a method of treating cancer. While not intending to be bound by theory, radiation therapy works by damaging the DNA of cells. The damage is caused by an electromagnetic, electron or proton beam directly or indirectly ionizing the atoms which make up DNA chain. Indirect ionization happens as a result of the ionization of oxygen, forming free radicals, which then damage the DNA. In the most common forms of radiation therapy, most of the radiation effect is through free radicals. Because cells have mechanisms for repairing DNA breakage, where the DNA is broken on both strands of the DNA are the most significant in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly. Radiation therapy can be used to treat almost every type of solid tumor, including cancers of the brain, breast, cervix, larynx, lung, pancreas, prostate, skin, spine, stomach, uterus, or other soft tissue sarcomas. Radiation can also be used to treat leukemias and lymphomas.

Radiation therapy includes the use of a variety of different types of radiation, as well as different methods of administering the radiation, and varying dosages of radiation. The choice of method and type of radiation best suited to a particular cancer can be determined by one skilled in the art. One type of radiation is electromagnetic radiation, which includes x-rays that have energies in the range 100 eV to 100 thousand electron volt (KeV), and γ-rays that generally have energies greater than 100 KeV. Normally, radiation is administered to subjects in the clinical setting using 2 million electron volt (MeV) to 10 MeV machines. Another type of radiation is particle beams, which are beams of fast-moving subatomic particles such as electrons, protons, neutrons, heavy ions, and pions. As particle beams often exhibit low penetration, they are preferably used to treat cancers located on the surface or just below the skin. The unit used to measure radiation dosages is the gray (Gy), which is the equivalent of 100 rads. The dosage used varies depending on a variety of variables, including the type of tissue being irradiated. For example, a human liver can tolerate a total dose of 3,000 cGy, while human kidneys can only tolerate about 1,800 cGy. Radiotherapy generally involves delivering multiple small fractions of radiation over time to reduce side effects.

Radiation therapy is preferably administered daily. The dose depends primarily on tumor type, but many other factors such as whether radiation is given before or after surgery, the success of surgery and its findings and many other reasons known by those skilled in the art. For radical (curative) cases, the typical dose for a solid epithelial tumor may range from about 50 to 70 grays (Gy) or more, while lymphomas (white cell) tumors might receive doses closer to about 20 to 40 Gy given in daily doses (a daily dose is a fraction); in adults these are typically 1.8 to 2 Gy per fraction. These small frequent doses allow healthy cells time to grow back, repairing damage inflicted by the radiation. The total dose can be given in daily fractions using external beam radiation or the total dose can be given via other methods such as implants that deliver radiation continuously over a given timeframe. The typical treatment schedule is 5 days per week. However, there are alternative fractionation schedules such as the CHART (Continuous Hyperfractionated Accelerated RadioTherapy) regimen for lung cancer, which used 2 or 3 smaller fractions per day, may also be used. In palliative cases, a single dose of 6-10 Gy may be given to painful superficial tumors (e.g., a rib metases) to relieve pain.

Methods of delivering radiation include external radiation therapy, internal radiation therapy, systemic radiation therapy, and prophylactic radiation therapy. External radiation therapy involves delivering radiation from a machine outside the body, and includes methods such as interoperative radiation therapy and 3-D conformal radiation therapy. Internal radiation uses radiation delivered from a machine, generally within an implant, that is placed internally, very close to or inside the tumor. Interstitial radiation therapy and intracavitary radiation therapy are examples of internal radiation therapy. Preferably, external and internal radiation are focused to primarily effect the target tissue, which includes the tumor being treated. Systemic radiation therapy involves delivering radioactive materials such as Iodine-131 (¹³¹I) or Strontium-89 (⁸⁹Sr) orally or by injection. Prophylactic radiation therapy is conducted to prevent a tumor from obtaining a foothold in a particular area, and typically involves the application of external radiation. In particular, prophylactic radiation therapy involves delivery of radiation to the brain when the primary cancer is highly metastatic (e.g., small cell lung cancer) and has a high risk of metastasizing to the brain.

Angiogenesis and Tumor Growth

Angiogenesis is the generation of new blood vessels in tissue. Tumor growth and metastasis have been shown to be angiogenesis dependent, and tumors unable to induce angiogenesis generally remain dormant at a microscopic in situ size. For example, in immunodeficient mouse models, heterotrasplanted malignant cells sometimes fail to form grossly-identifieable tumor nodules, but nonetheless persist as small, non-angiogenic tumors called “no-takes.” See Achilles et al., J. Natl. Cancer Ins. 93, 1075-1081 (2001). While not intending to be bound by theory, cancer cells that are unable to simulate angiogenesis do not appear to be able to obtain sufficient oxygenation and other nutrients needed for aggressive cell proliferation. Thus, while cancer is caused by cells that exhibit abnormal and excessive cell proliferation, the mere presence of tumor cells may not be sufficient in many cases to cause cancer, and the tumor cells may remain relatively harmless unless they are able to stimulate pathological angiogenesis to support their growth.

Previous research described in Griffioen et al., Blood 88, 667-673 (1996), and Griffioen et al., Cancer Res. 56, 1111-1117 (1996) has shown that pro-angiogenic factors in tumors induce down-regulation of adhesion molecules on endothelial cells in the tumor vasculature and induce anergy to inflammatory signals such as tumor necrosis factor-α (TNFα), interleukin-1, and interferon-γ. Endothelial cells (EC) exposed to vascular endothelial cell growth factor (VEGF) have a severely hampered up-regulation of intercellular adhesion molecule-1 (ICAM-1) and induction of vascular cell adhesion molecule-1 (VCAM-1) and E-selectin. This phenomenon, referred to as tumor-induced EC anergy, is one way in which tumors with an angiogenic phenotype may escape infiltration by cytotoxic leukocytes.

Because angiogenesis-mediated down-regulation of endothelial adhesion molecules (EAM) may promote tumor outgrowth by avoiding the immune response (Griffioen et al., Blood 88, 667-673 (1996); Kitayama et al., Cancer. Res. 54 4729-4733 (1994); and Piali et al., J. exp. Med. 181, 811-816 (1995)), it is believed that inhibition of angiogenesis would overcome the down-regulation of adhesion molecules and the unresponsiveness to inflammatory signals. In support of this hypothesis, a relation between E-selectin up-regulation and the angiostatic agent AGM-1470 has been reported (Budson et al., Biochem. Biophys. Res. Comm. 225, 141-145 (1996)). It has also been shown that inhibition of angiogenesis by PF4 up-regulates ICAM-1 on bFGF-simulated EC. In addition, inhibition of angiogenesis by PF4 overcomes the angiogenesis-associated EC anergy to inflammatory signals.

Accordingly, one aspect of tumor treatment by the combination of β-sheet peptides and radiotherapy of the invention includes inhibiting angiogenesis and endothelial cell proliferation, as described further herein.

Tumor Treatment by β-sheet Peptides and Radiation Therapy

The β-sheet peptides of the invention can be administered to a patient (e.g., a mammal such as a human) in conjunction with radiation therapy as a method of treating cancer. In conjunction, as used herein, refers to administration of the β-sheet peptides either before, after, or during radiation therapy, but sufficiently proximate in time such that the effects of the two treatment modalities overlap. For example, in one embodiment, β-sheet peptides may be administered before delivery of radiation. In another embodiment, β-sheet peptides may be administered after delivery of radiation. Examples 4 and 6, below, for instance, demonstrate the radiosensitizing activity of β-sheet peptides delivered prior to administration of radiation, while Example 9 demonstrates the synergistic effect of β-sheet peptides delivered after administration of radiation.

While the pharmacokinetics of individual β-sheet peptides vary to some degree, pharmacokinetic studies have demonstrated that β-sheet peptides can persist in the vasculature surrounding a tumor site for several days. For example, administration of β-sheet peptides 24 hours before or after administration of radiation is sufficiently proximate in time such that the effects of the two treatment modalities overlap. More preferably, the β-sheet peptides are administered within 12 hours of radiation therapy. The dosage of β-sheet peptide administered will vary in response to a variety of factors such as the tumor size and location, the size of the subject, and the means of administration. Determination of an appropriate dosage can be readily determined based on those generally used for chemokine peptides by one skilled in the art. However, due to the efficacy of the β-sheet peptides, lower dosages may be suitable as well.

The cancer treated by the method of the invention may be any of the forms of cancer known to those skilled in the art or described herein. Cancer that manifests as both solid tumors and cancer that instead forms non-solid tumors as typically seen in leukemia can be treated.

Treatment, as defined herein, is a reduction in tumor load or decrease in tumor growth in a patient in response to the administration of β-sheet peptides and radiation therapy. The reduction in tumor load may be represent a direct decrease in mass, or it may be measured in terms of tumor growth delay, which is calculated by subtracting the average time for control tumors to grow over to a certain volume from the time required for treated tumors to grow to the same volume. The patient is preferably a mammal, such as a domesticated farm animal (e.g., cow, horse, pig) or pet (e.g., dog, cat). More preferably, the patient is a human.

Cancer treatment by the β-sheet peptides used in conjunction with radiation therapy preferably results in a synergistic therapeutic effect. A synergistic effect, as defined herein, occurs when treatment by a β-sheet peptide in conjunction with radiation therapy results in a reduction in tumor load or growth delay that is greater than the reduction in tumor load or growth delay that is observed when the effects of separate treatment by radiation therapy and the β-sheet peptides of the invention are added together, where the radiation and β-sheet peptides dosages and treatment schedules are otherwise the same when used individually or in combination. The comparison of the combined treatment with the effects of separate treatment, added together, result in a ratio that will be greater than 1 (i.e., greater than 100%) if a synergistic effect is present. Preferably, a synergistic effect with a ratio of at least 2 (i.e., at least 200%) is provided by the method of the invention, and more preferably the synergistic effect has a ratio of at least 3 (i.e., at least 300%). For further discussion of the determination of a synergistic effect, see Example 3, herein.

β-sheet peptides used in the method of the present invention may exhibit activity as radiosensitizing agents. A radiosensitizing agent, as defined herein, is a substance that increases the sensitivity of tumor cells to damage by radiation therapy. Radiosensitization may occur either by directly by increasing the susceptibility of tumor cells to damage by radiation, or by hindering the ability of tumor cells damaged by radiation to repair the damage inflicted. An agent that functions strictly as a radiosensitizing agent will have no significant effect on a tumor when used alone, but will exhibit a substantial effect on tumor growth and/or load when used in conjunction with radiation therapy. For instance, in Example 8, βpep-25 demonstrated little effect on tumor growth when used alone. Likewise, radiation treatment alone produced an incomplete response. However, use of βpep-25 in combination with radiation significantly increased tumor growth delay. Example 9 demonstrates that βpep-25 is able, in a further aspect of the invention, to radiosensitize the response of endothelial cells to radiation.

Cancer treatment by the β-sheet peptides of the present invention may include a variety of specific effects on tumor tissue. For instance, β-sheet peptides of the present invention may exhibit activity as angiogenesis inhibitors. An angiogenesis inhibitor is a substance that decreases angiogenesis, as described herein. For instance, in Example 10, βpep-14 and βpep16 were shown to decrease endothelial cell (EC) proliferation in vitro, which is a standard method in the art of demonstrating the efficacy of a substance as an angiogenesis inhibitor. In a further aspect of their activity as angiogenesis inhibitors, βpep-14 and βpep-16 are able to prevent fibroblast growth factor (bFGF) mediated downregulation of intercellular adhesion molecule-1 (ICAM-1). In FIG. 11, βpep1-24 were all shown to have activity as angiogenesis inhibitors. Example 10 further provides methods for testing the angiogenesis or endothelial proliferation inhibiting capacity of β-sheet peptides suitable for testing the efficacy of β-sheet peptides used in the present method. In the case of tumor angiogenesis, it may be preferable to measure the response to tumor microvessel density (MVD) to treatment. MVD indicates the vascularization of a tumor that results from tumor angiogenesis, and it is expected to diminish in response to treatment with an angiogenesis inhibitor. In Example 5, βpep-25 infusion was demonstrated to decrease MVD, again supporting its activity as an angiogenesis inhibitor. Thus, an additional aspect of the method is the ability to reduce tumor microvessel density.

Further aspects of the method relate to the ability of β-sheet peptides to effect tumor physiology. Aspects of tumor physiology include, for instance, tumor oxygen levels (pO₂) and tumor blood perfusion. Preferably, cancer treatment by β-sheet peptides used in conjunction with radiation therapy will result in a reduction of tumor oxygen levels and/or tumor blood perfusion. While not intending to be bound by theory, reduction of tumor oxygen levels and/or blood perfusion indicate a diminished ability of a tumor to receive the materials it needs for continued cell proliferation, which leads to an inhibition of tumor growth. Preferably, tumor oxygen levels and/or blood perfusion levels are decreased to a level at which tumor cells are not only prevented from proliferating, but in addition become subject to hypoxia and necrosis.

Mechanism of Radiosensitization by β-sheet Peptides

While not intending to be bound by theory, the potential mechanism of action of β-sheet peptides warrants discussion. First, the effects of β-sheet peptides should be distinguished from the effects of radiation alone. Radiation treatment alone led to a 50% decrease in microvessel density (MVD) in MA148 tumors. This is in agreement with recent work of Garcia-Barros et al. who reported that the primary effect of radiation on the tumor is via endothelial cells (Garcia-Barros et al., Science 300, 1155-59 (2003)). However, the faster growing SCK tumors treated solely by radiation showed no significant change in MVD. This was surprising because the baseline MVD in SCK control tumors was twice as high as that in MA148 control tumors, and it was expected that the Garcia-Barros et al. hypothesis would hold. It must be remembered that the kinetics of tumor cell division and tumor cell loss in a given tumor type remain an important factor in the response to treatment, which may or may not correlate to MVD. Nevertheless, the combination of βpep-25 and radiation remarkably decreased MVD and increased treatment response in SCK tumors compared to stand-alone therapy. This was clearly demonstrated in SCK tumors where there was a synergistic decrease in MVD, whereas in the MA148 model this was implied because all tumors disappeared completely by the end of the treatment period.

Because MVD in tumors was reduced more by combination therapy, it was expected that combination therapy would also cause an increase in viability (parenchymal and stromal cells). However, this was not the case in SCK mammary carcinoma tumors (at least when measured after 14 days of anginex infusion and a single dose of 25 Gy), where only cell proliferation was highly attenuated. Moreover, immunohistochemical colocalization (TUNEL or PCNA with anti-CD31 antibody to locate vessels) also revealed that both SCK and MA148 ovarian carcinoma tumors in control mice had a greater number of proliferating endothelial cells (EC) and fewer apoptotic EC compared to EC in tumors from any of the treated groups. This provides further indication that β-sheet peptides, as well as radiation, disrupt the function of EC in tumors.

While not intending to be bound by theory, the ability of β-sheet peptides to specifically target EC in newly forming blood vessels in tumors appears to make tumor tissue more susceptible to radiation and reduces the ability of tumors to recover from radiation, thereby explaining the results of the tumor growth delay assays presented in the Examples. The Examples further indicate that β-sheet peptides sensitize EC to radiation treatment. For instance, the in vitro experiments illustrate the specific effects from βpep-25 (limited 4 hour exposure) and radiation on EC proliferation and on colony formation, but not on either tumor cell line (SCK or MA148). This is further validated by the fact that after only three daily i.p. injections of βpep-25, the response of SCK tumors to radiation is significantly improved, with half of the tumors completely regressing. Interestingly, no tumor regressions were observed using the same protocol with angiostatin (Mauceri et al., Nature 394, 287-91 (1998)), another antiangiogenic agent that operates via a molecular mechanism or target different from βpep-25. Pharmacologically, the half-life of βpep-25 in mice is on the order of 50 to 90 minutes. As an antiangiogenic agent, the effects of βpep-25 on tumor growth are observed only following several days of treatment (Dings et al., Cancer Lett. 194, 55-66 (2003)). In combination with radiation, however, effects from βpep-25 are observed on a much shorter time scale. Previous studies with βpep-25 demonstrated that this peptide functions on a shorter time scale by inhibiting EC adhesion to and migration on the extracellular matrix in vitro (Griffioen et al., Biochem J. 354, 233-42 (2001)). This, in turn, suggests that β-sheet peptides function this way in vivo as well. In all, the fact that angiostatin had little if any effect on the SCK tumor radiation response while βpep-25 produced a significant response is encouraging.

Although MVD and changes in vascular patterns are commonly used markers of anti-angiogenic efficacy, assessing other physiological parameters can be another valuable way to assess anti-angiogenic efficacy. A time-dependent increase in tumor pO₂, or blood flow upon treatment with antiangiogenic agents (Kozin et al., Cancer Res. 61, 39-44 (2001)), or inhibition of VEGF-induced protection against, and/or repair of, radiation damage in EC (Reinmuth et al., Faseb J. 15, 1239-41 (2001)) have been suggested as possible mechanisms by which anti-angiogenic agents enhance radiation response. Previously, it was determined that a single injection of SU6668 transiently decreased tumor blood perfusion and permanently reduced tumor perfusion after 1-2 weeks of daily injections of SCK-bearing animals (Griffin et al., Cancer Res. 62, 1702-06 (2002)). The present Examples show that daily injections of βpep-25 result in reduced blood flow and tumor oxygenation, without affecting blood pressure, in size-matched tumors not exceeding 1000 mm³. These data suggest that the time of assessment is crucial to observe the true effects from anti-angiogenic and/or radiation therapy. Another study (Lee et al., Cancer Res. 60, 5565-70 (2000)) reported a similar finding for monitoring the effects of endostatin treatment. The fact that large experimental tumors commonly have widespread hypoxia and necrosis, even without any treatment, may also explain why it is difficult, if not impossible, to detect differences in physiology between vehicle-treated mice and anginex-treated mice at extended time points with late stage tumors. However, the fact that it is possible to detect changes in tumor physiology during the early stages of treatment supports the clinical use of similar markers monitored via functional MRI or other non-invasive imaging methods. This type of analysis has already been demonstrated in a phase I human colon cancer trial with bevacizumab (Avastin) (Willett et al., Nat. Med. 10, 145-47 (2004)).

Example 9 supplements the understanding of β-sheet peptides effects by demonstration that β-sheet peptides can inhibit re-vascularization of tumor tissue following radiation damage, and avoid inducing hypoxia before radiation therapy is given. Combination therapy did indeed cause a greater delay in SCCVII tumor growth compared to βpep-25 or radiation therapy alone. The delay was 10 days post treatment, and tumor size was maintained at the same level for approximately one week thereafter.

Example 9 demonstrates that low-dose administration of βpep-25 following irradiation delays tumor growth, indicating that using an angiogenesis inhibitor may be effective as an adjuvant therapy after completion of radiotherapy. It has also been suggested that radiotherapy, antitumor chemotherapy or their use in combination facilitates tumor disappearance macroscopically after surgery, and that intermittent administration of an angiogenesis inhibitor inhibits local recurrence of the tumor. The choice of treatment protocol depends on the intrinsic characteristics of the tumor. The results above demonstrate that βpep-25 given just prior to radiation was more effective than angiostatin at increasing the response to radiation of the highly aggressive SCK breast carcinoma in our previous study. SCK tumors are very hypoxic, and thus addition of anginex before radiation probably did little to influence oxygen-mediated radiation cell killing. Hence, a complete response of the tumor may be achieved by using radiotherapy in combination with a β-sheet peptide administered at a relatively high dose before or after radiation. Example 9 highlights the use of adjuvant therapy using an angiogenesis inhibitor to target re-vascularization of the primary tumor and local recurrence following radiation-induced tumor reduction.

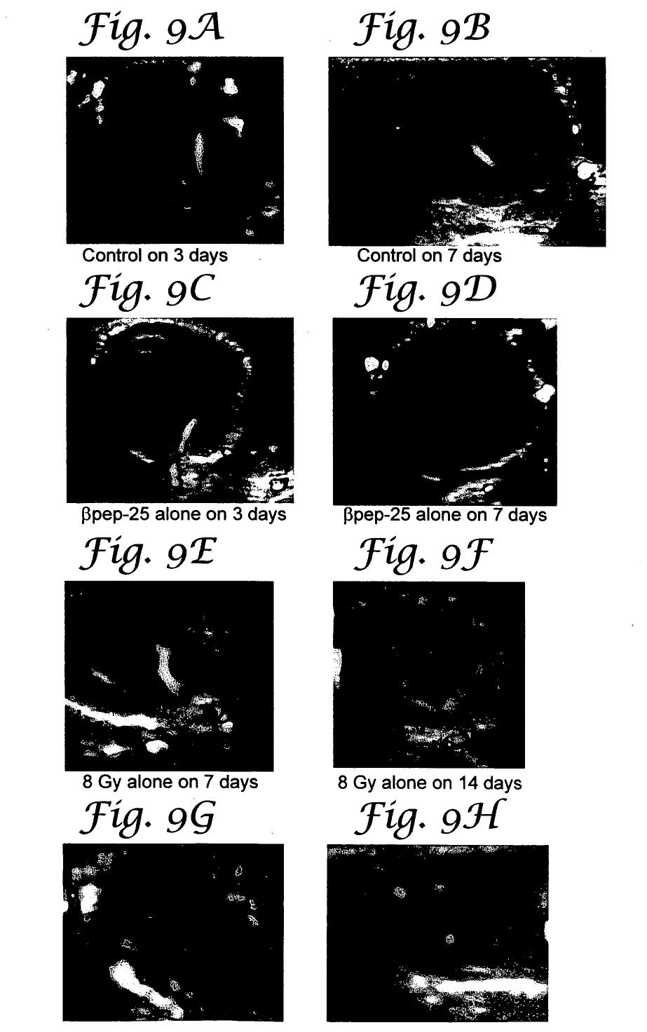

While not intending to be bound by theory, the results from the sonography studies, shown in FIG. 9, provided some mechanistic insight into why β-sheet peptides, in combination with radiation therapy, are so effective. Sonography is a rapidly evolving and potentially powerful imaging method for experimental analysis of human cancer. The ability to longitudinally image tumor vasculature before, during and after therapy is an extremely valuable tool in assessing effects from treatment (Weller et al., Cancer Res. 65(2), 533-9 (2005)). Blood flow in vessels of tumors from βpep-25-treated mice was decreased one week after treatment, compared with those in control mice. This strongly suggests that a decrease in blood supply to the tumor was causal to delaying tumor growth. Griffioen et al. reported that in the sprout-formation assay using bovine capillary endothelial cells (ECs), βpep-25 did not affect resting ECs in confluent monolayers, but did affect actively growing EC. This suggests that βpep-25 should not act on quiescent EC in normal vasculature in vivo. Moreover, anginex was reported to have a specific toxic effect on endothelial cells, and very little, if any, toxic effect MA148 tumor cells (Dings et al., Cancer Lett. 194, 55-66 (2003)). This is confirmed by Example 9, which shows that SCCVII tumor cell growth is also not affected by the presence of βpep-25. Therefore, the effect from βpep-25 in the in vivo studies with SCCVII tumors is likely through endothelial cells. Power doppler imaging supports this, and allowed in vivo observation of the specific cytotoxic effect from βpep-25 on activated intra-tumor vessels. This effect is correlated to the observed depression of the oxygen partial pressure in tumors and an increase in the hypoxic fraction.

Overall, the Examples clearly demonstrate that β-sheet peptides in combination with radiotherapy are effective at inhibiting tumor progression in animal models. This observation, combined with the general absence of toxicity alone or in combination with radiation, underscores the clinical potential of these compounds.

Administration and Formulation of β-sheet Peptides

The β-sheet peptides of this invention can be administered alone in a pharmaceutically acceptable carrier, as an antigen in association with another protein, such as an immunostimulatory protein or with a protein carrier such as, but not limited to, keyhole limpet hemocyanin (KLH), bovine serum albumin (BSA), ovalbumin, or the like. They may be employed in a monovalent state (i.e., free peptide or a single peptide fragment coupled to a carrier molecule). They may also be employed as conjugates having more than one (same or different) peptides bound to a single carrier molecule. The carrier may be a biological carrier molecule (e.g., a glycosaminoglycan, a proteoglycan, albumin or the like) or a synthetic polymer (e.g., a polyalkyleneglycol or a synthetic chromatography support). Typically, ovalbumin, human serum albumin, other proteins, polyethylene glycol, or the like are employed as the carrier. Such modifications may increase the apparent affinity and/or change the stability of a peptide. The number of peptides associated with or bound to each carrier can vary, but from about 4 to 8 peptides per carrier molecule are typically obtained under standard coupling conditions.

The β-sheet peptides can be conjugated to other polypeptides using standard methods such as activation of the carrier molecule with a heterobifunctional sulfosuccinimidyl 4-(n-maleimidomethyl)cyclohexane-1-carboxylate reagent. Cross-linking of an activated carrier to a peptide can occur by reaction of the maleimide group of the carrier with the sulfhydryl group of a peptide containing a cysteine residue. Conjugates can be separated from free peptide through the use of gel filtration column chromatography or other methods known in the art.

For instance, peptide/carrier molecule conjugates may be prepared by treating a mixture of peptides and carrier molecules with a coupling agent, such as a carbodiimide. The coupling agent may activate a carboxyl group on either the peptide or the carrier molecule so that the carboxyl group can react with a nucleophile (e.g., an amino or hydroxyl group) on the other member of the peptide/carrier molecule, resulting in the covalent linkage of the peptide and the carrier molecule.

For example, conjugates of a peptide coupled to ovalbumin may be prepared by dissolving equal amounts of lyophilized peptide and ovalbumin in a small volume of water. In a second tube, 1-ethyl-3-(3-dimethylamino-propyl)-carboiimide hydrochloride (EDC; ten times the amount of peptide) is dissolved in a small amount of water. The EDC solution is added to the peptide/ovalbumin mixture and allowed to react for a number of hours. The mixture may then be dialyzed (e.g., into phosphate buffered saline) to obtain a purified solution of peptide/ovalbumin conjugate.

The present invention also provides a composition that includes one or more active agents (i.e., β-sheet peptides) of the invention and one or more pharmaceutically acceptable carriers. One or more β-sheet peptides with demonstrated biological activity can be administered to a patient in an amount alone or together with other active agents and with a pharmaceutically acceptable buffer. The β-sheet peptides can be combined with a variety of physiological acceptable carriers for delivery to a patient including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a patient, preferably a mammal, and more preferably a human, the composition of the invention in an amount effective to produce the desired effect. The peptides can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The agents of the present invention are preferably formulated in pharmaceutical compositions and then, in accordance with the methods of the invention, administered to a patient, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, and intravenous) administration.

Formulations suitable for parenteral administration conveniently include a sterile aqueous preparation of the active agent, or dispersions of sterile powders of the active agent, which are preferably isotonic with the blood of the recipient. Parenteral administration of β-sheet peptides (e.g., through an I.V. drip) is a preferred form of administration. Isotonic agents that can be included in the liquid preparation include sugars, buffers, and sodium chloride. Solutions of the active agent can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectible solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the active agents over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active agent as a powder or granules, as liposomes containing the chemopreventive agent, or as a solution or suspension in an aqueous liquor or non-aqueous liquid such as a syrup, an elixir, an emulsion, or a draught. Such compositions and preparations typically contain at least about 0.1 wt-% of the active agent. The amount of β-sheet peptide (i.e., active agent) is such that the dosage level will be effective to produce the desired result in the patient.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Gamma and X-ray Irradiation

For X-ray irradiation, mice were first anesthetized and covered with a 4 millimeter (mm) thick lead shield. The tumor-bearing legs were gently extended into the radiation field and exposed to X-rays at a dose rate of 1.4 Gy/minute. A Philips 250 kV orthovoltage machine (Philips Medical Systems, Brookfield, Wis.) was used for the irradiation (Griffin et al., Cancer Res 62, 1702-06 (2002)). In the in vitro experiments cells were irradiated with γ-rays using a ¹³⁷Cs irradiator (Mark I Cesium irradiator; J. L. Shepherd and Associates, Glendale, Calif.) at a dose rate of 0.9 Gy/minute.

### Example 2

### Carcinoma Mouse Models and Radiosensitization Test

To prepare the MA148 ovarian carcinoma mouse model, female athymic nude mice (nu/nu, 5-6 weeks old) were purchased from the National Cancer Institute and allowed to acclimate for one week. Exponentially growing human ovarian MA148 epithelial carcinoma cells were cultured, harvested, suspended in serum free RPMI (2.0×10⁷ cells/milliliter (ml)) and inoculated subcutaneously (s.c.) into the right flank of the mouse (Dings et al., Cancer Res 63, 382-85 (2003)). Treatment was initiated after randomization of mice, when established tumors reached a size of at least 50 mm³. Four groups were assigned, control (n=13), βpep-25 (n=10), radiation (n=10), and the combination of βpep-25 and radiation (n=10). βpep-25, dissolved in PBS was administered using osmotic mini-pumps (10 milligrams (mg)/kilograms (kg)/day; Durect; Cupertino, Calif.) implanted s.c. into the left flank. The pumps had a treatment span of 28 days. The mice of the control group were given PBS administered with mini-pumps. Tumors were locally irradiated with 5 Gy once a week for 4 weeks.

To prepare the SCK murine mammary carcinoma model, SCK tumor cells, 2×10⁵ cells in 0.05 ml of serum-free medium, were injected s.c. into the right hind thigh of male A/J mice (Griffin et al., Cancer Res 62, 1702-06 (2002)). The tumors were allowed to grow to 125 mm³ in volume. After randomization four groups were assigned, control (n=12), βpep-25 (20 mg/kg; n=12), radiation (n=13) and the combination of βpep-25 and radiation (n=13). βpep-25 treatment (20 mg/kg) was initiated utilizing 14-day osmotic mini-pumps. Radiation (25 Gy) was given once, two days after initiation of βpep-25 treatment.

To test for radiosensitization of the tumor endothelium, SCK tumors at a volume of 200-225 mm³ were treated with intraperitoneal (i.p.) injections of βpep-25 (20 mg/kg) or angiostatin (25 and 50 mg/kg) on days −1, 0 and 1. On days 0 and 1 the injections were given 2 hours prior to tumor irradiation (10 Gy each day) (Mauceri et al., Nature 394, 287-91 (1998)). Human angiostatin was produced from human plasma (Kirsch et al., Cancer Res. 58, 4654 (1998)). The treatment groups each contained 6 or 10 tumor-bearing mice in the βpep-25 or angiostatin studies, respectively.

Tumor volume was determined by measuring the diameters of the tumor using metric-scale calipers (Scienceware; Pequannock, N.J.) and solving the equation to determine the volume of a spheroid: (a²×b×Π)/6, where ‘a’ is the width and ‘b’ is the length of the tumor.

### Example 3

### Statistical Analysis and Synergy Determination

Data sets were analyzed using a commercially available software package (InStat 2.03, Graphpad Software, Inc.). A two-tailed Student's t test was used to determine the validity of the differences between control and treatment data sets. A P value of 0.05 or less was considered significant.

In order to determine the degree to which the combination of βpep-25 and radiation had synergistic effects on tumor growth delay the following formula was applied: observed growth delay from combination treatment/{(tumor growth delay from treatment 1)+(tumor growth delay from treatment 2)}. A ratio of greater than 1 indicates a synergistic (greater than additive) effect. Growth delay was calculated by subtracting the average time for control tumors to grow three-fold in volume from the time required for treated tumors to increase in volume by 3-fold from the day of radiation.

### Example 4

### βpep-25 Potentiates Radiation Therapy

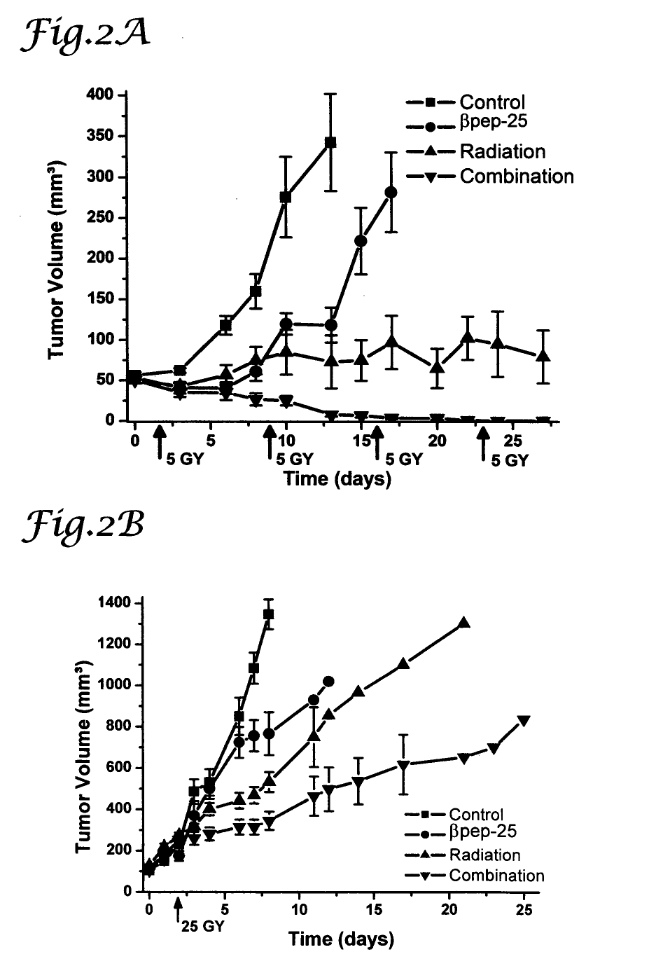

The potentiation of radiation therapy by βpep-25 was initially demonstrated by administering βpep-25 to mice bearing established human MA148 ovarian tumor xenografts, in combination with 5 Gy once per week for 4 weeks. Tumors were allowed to grow to about 50 mm³ and animals were treated concurrently with βpep-25 (systemically via implanted osmotic mini-pumps) and radiation treatment (once weekly). After 2 weeks of treatment with βpep-25 alone, tumor growth was inhibited up to 70% (tumor volume) compared to control, with an average delay in tumor growth of 7 days (FIG. 2a). Radiation treatment resulted in an effective reduction of tumor growth leading to tumor stasis; however, when radiation was combined with βpep-25, no tumor mass could be detected at the end of 4 weeks of continuous βpep-25 infusion in these mice (FIG. 2a). However, regrowth of tumors was observed two weeks post termination of treatment.

In the study employing the SCK murine mammary carcinoma grown in immunocompetent mice, treatment was initiated on established tumors of 125 mm³ in volume. Anticipating the aggressiveness of this tumor model, βpep-25 was given at a dose of 20 mg/kg and a single dose of 25 Gy radiation was given on day 2. Control tumors grew to 3-fold of the average volume on the day of radiation exposure for the other groups in 4 days. Tumors of mice being treated with βpep-25 also grew three-fold in volume in 4 days (FIG. 2b), whereas radiation treatment alone delayed tumor growth by 5 days. Combination treatment, on the other hand, resulted in a 16 days tumor growth delay compared to control. The synergy ratio was calculated as [tumor growth delay caused by the combination of βpep-25 and 25 Gy (growth delay of 16.0 days)]/[growth delay by βpep-25 (growth delay of 0 days)+growth delay by 25 Gy (growth delay of 5 days)]=3.2. Therefore, combination treatment had a synergistic effect on tumor growth delay.

### Example 5

### Histological Analysis of Microvessel Density, Cell Death and Proliferation

Evaluation of immunohistochemistry was carried out as follows. MA148 ovarian xenografts were excised at the end of 28 days of βpep-25 infusion. In the SCK mouse model, tumors were taken excised after 14 days of βpep-25 infusion, or the day at which the animal was deemed too weak to survive another day and was sacrificed. Similar size tumors without apparent widespread necrosis were embedded in tissue freezing medium (Miles Inc.; Elkart, Ind.) and snap frozen in liquid nitrogen. Preparation and procedures were done as described by Wild et al. (Microvasc. Res. 59, 368 (2000)). Samples were subsequently incubated in a 1:50 dilution with phycoerythrin (PE)-conjugated monoclonal antibody to mouse CD-31 (PECAM-1) (Pharmingen; San Diego, Calif.) or a fluorescein isothiocyanate (FITC)-conjugated PCNA (Ab-1) (Oncogene; San Diego, Calif.) to stain for microvessel density (MVD) or proliferation, respectively. At the same time the sections were also stained for cell death using a TUNEL (terminal deoxyribonucleotidyl transferase-mediated dUTP-nick-end labeling) assay carried out according to the manufacturer's instructions (in situ cell death detection kit, fluorescein; TUNEL, Roche). The vessel density and architecture, as well as the proliferation and apoptosis was quantified (Dings et al, Cancer Lett., 194, 55 (2001); Wild et al., Microvasc. Res. 59, 368 (2000)) and summarized in Table 2.

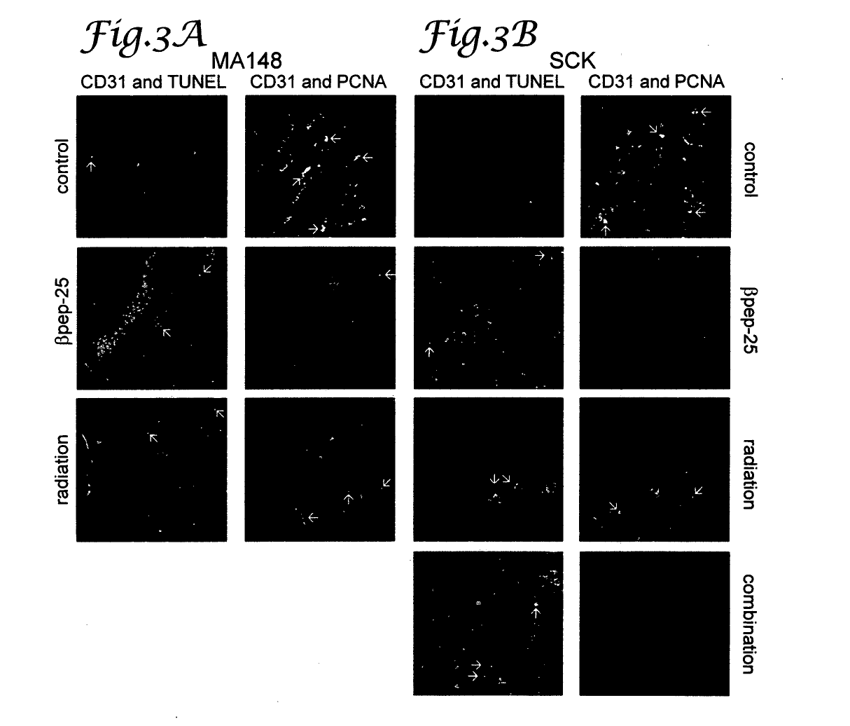

In the MA148 tumors, βpep-25 infusion at 10 mg/kg/day for 28 days resulted in a decrease of tumor microvessel density (MVD), as determined by CD31 staining, indicating that the anti-tumor activity of βpep-25 is the result of angiogenesis inhibition. Table 2 and FIG. 3 summarize the immunohistochemical study and the results from digital analysis of stained tissue sections. Radiation exposure also reduced MA148 MVD by a substantial margin. MA148 tumors treated with the combination of βpep-25 and radiation could not be stained because all tumors had regressed at the end of the 28 days of βpep-25 infusion and 4 weekly doses of 5 Gy. Aside from vessel density (including number, size and length), the digital approach discriminates vessel branch points, end points and vessel lengths. Changes in these architectural parameters were revealed by this digital method (Table 2). For example, the vessel length and the amount of end points of the vessels in tumors of animals treated with either βpep-25 or radiation were convincingly less (P less than 0.001) than that in control tumors. This trend was also observed with the amount of cells undergoing proliferation, as determined by PCNA staining. Tumors treated with either βpep-25 or radiation alone showed only about half the proliferation of control tumors (P less than 0.01; Table 2), with an insignificant increase in total cell death (stromal and parenchymal cells), as determined by the TUNEL method.

In SCK tumors, neither βpep-25 nor radiation treatment alone significantly affected MVD in tumors of treated animals compared to those of controls. However, combination treatment did cause a decrease in MVD compared to control (P less than 0.001) and βpep-25 treatment (P less than 0.01). In addition, combination treatment also affected vessel architecture (Table 2), for example, by significantly reducing the number of end points and branch points (P less than 0.001 and P less than 0.01 respectively). On the other hand, combination treatment did not enhance total cell death compared to either stand-alone therapy, whereas it did show a significant difference in total cell death compared to control tumors (P less than 0.01, Table 2).

Because TUNEL and PCNA staining are non-specific for cell type, double staining was performed in conjunction with CD31 staining. This revealed that tumors in control mice had a greater amount of proliferating endothelial cells and fewer endothelial cells undergoing apoptosis compared to βpep-25, radiation, or combination-treated tumors. This trend was observed in both tumor models (FIG. 3).

TABLE 2
HISTOLOGICAL ANALYSIS OF MICROVESSEL DENSITY, CELL
DEATH AND PROLIFERATION

| | Vessel | End | Branch | Vessel | | |
|---|---|---|---|---|---|---|
| | Density¹ | Points² | Points³ | Length⁴ | Proliferation | Apoptosis |
| MA148 | | | | | | |
| Vehicle | 11094 ± 1173.4 | 40.2 ± 3.5 | 2.7 ± 0.62 | 7.93 ± 0.66 | 3561 ± 405 | 1543 ± 917 |
| βpep-25 | 5105 ± 606.9⁵ | 18.8 ± 2.6⁵ | 1.5 ± 0.82 | 4.22 ± 0.66⁵ | 1255 ± 311⁶ | 2407 ± 840 |
| Radiation | 5540 ± 704.8⁵ | 18.6 ± 2.3⁵ | 1.2 ± 0.99 | 4.36 ± 0.79⁵ | 1612 ± 279⁶ | 2130 ± 471 |
| SCK | | | | | | |
| Vehicle | 23452 ± 1762 | 68.1 ± 7.6 | 4.1 ± 1.3 | 13.7 ± 1.5 | 14637 ± 4323 | 175 ± 82 |
| βpep-25 | 20094 ± 1968 | 66.0 ± 5.6 | 5.1 ± 1.4 | 11.0 ± 1.6 | 2707 ± 272⁶ | 2177 ± 395⁶ |
| Radiation | 23305 ± 2877 | 49.8 ± 14.9 | 9.9 ± 2.5 | 14.9 ± 3.1 | 2257 ± 220⁶ | 1767 ± 788⁶ |
| Combination | 8045 ± 586^{5,7} | 33.4 ± 2.7^{5,7} | 1.1 ± 0.4^{6,7} | 5.5 ± 0.7^{5,7} | 1254 ± 423⁶ | 1115 ± 314⁶ |

¹Following binarization of images (magnification 200X), microvessel density was estimated by scoring the total number of white pixels per field. Results show the mean white pixel count per image ± standard error.
²Mean number of vessel end points ± standard error as determined after skeletonization of the images.
³Mean number of vessel branch points/nodes per image in pixels ± standard error as determined after skeletonization of the images.
⁴Mean total vessel length per image in pixels ± standard error as determined after skeletonization of the images.
⁵P less than 0.001. Experimental group compared to vehicle, using the Student's T test.
⁶P less than 0.01. Experimental group compared to vehicle, using the Student's T test.
⁷P less than 0.05. Experimental group compared to βpep-25, using the Student's T test.

### Example 6

### Absence of Toxicity from βpep-25 Treatment

As an indirect measurement of general toxicity, body weights of mice were monitored twice weekly, using a digital balance (Ohaus Florham, N.J.). To determine hematocrit and creatinine levels, blood samples were extracted by tail vein bleedings on the last day of treatment and blood was collected in heparinized micro-hematocrit capillary tubes (Fisher; Pittsburgh, Pa.). For hematocrit levels, samples were spun down for 10 minutes in a micro-hematocrit centrifuge (Clay-Adams; NY), and the amount of hematocrit was determined using an international microcapillary reader (IEC; Needham, Mass). To obtain creatinine levels, a kit was purchased from Sigma (Sigma Diagnostics; St Louis, Mo.) and used according to the manufacturer's instructions.

Animals treated with βpep-25 (alone or in combination with radiation) showed no signs of toxicity as assessed by unaltered behavior, weight gain during experiments, normal hematocrit and creatinine levels, and macro- and microscopic morphology of internal organs on autopsy. Body weights of mice were monitored as an indirect measurement of general toxicity. In experiments where tumors were irradiated, weights of mice halted initially and subsequently increased on termination of radiation treatment. βpep-25 did not augment this toxicity. In the xenograft model on the last day of treatment, blood was drawn and hematocrit and creatinine levels were determined as a measure of bone marrow and kidney toxicity, respectively. Hematocrit levels reported as a percentage of red blood cells (vehicle 49.0±3.5, βpep-25 49.6±2.5, radiation 49.6±0.6, and combination 51.0±1.0) and creatinine levels reported in μmoles/l (vehicle 49.2±6.1, βpep-25 48.0±5.0, radiation 48.5±1.4, and combination 44.4±1.4) showed no significant differences among treatment groups. The study with SCK tumors in immune competent mice showed similar hematocrit levels (vehicle 49.6±0.6, βpep-25 47.6±1.5, radiation 47.6±0.6, and combination 47±2.6 in percentage red blood cells) and creatinine levels (vehicle 38.4±8.2, βpep-25 40.7±5.3, radiation 39.9±12.0, and combination 39.4±10.0 in μmoles/l).

### Example 7

### βpep-25 Affects Tumor Physiological Function

Various assays were used to determine the effects of βpep-25 on tumor physiology functions. The blood perfusion in SCK tumors was measured with the ⁸⁶RbCl uptake method (Lin et al., Cancer Res. 53, 2076 (1993)). Anesthetized mice were injected with 5 μCi of ⁸⁶RbCl in 0.1 ml of PBS (pH 7.5) through the lateral tail vein, and sacrificed 60 seconds later by cervical dislocation. The tumors were removed, weighed, and the radioactivity was counted with a well-type gamma counter (1282 Compugamma; Pharmacia LKB Wallac, Turku, Finland). From the radioactivity in the tissue sample and that in the reference, the percentage of injected ⁸⁶RbCl per gram of tissue was calculated.

The pO₂ of tumors was measured with an Eppendorf pO₂ Histograph (Eppendorf, Hamburg, Germany. A pO₂ electrode (300 mm diameter) was inserted 1-2 mm deep by hand into the SCK tumors through small incisions made in the skin over the distal side of the tumor. The electrode was then advanced by a computer-controlled system measuring pO₂ along the track: the electrode was advanced by 0.7 mm forward steps, immediately withdrawn by 0.3 mm to reduce pressure artifact and the pO₂ value recorded. A total of 5 tracks were measured in each tumor, resulting in approximately 50 readings/tumor.

The blood pressure in awake mice was measured using a tail-cuff rodent blood pressure unit (Gilson Medical Electronics, Middleton, Wis.). Briefly, a Gould P23ID pressure transducer was put on the tail of a mouse that had been warmed on a 40° C. warming pad for 30 minutes (to enhance the signal in the tail via vasodilatation). The pressure transducer was connected to an ICT-2H chart recorder via an A-4023 conditioning amplifier. The cuff was inflated and released and systolic pressure was read by interpolating the pressure reading against a standard curve created with a manometer. See O'Bryan et al., J. Am. Soc. Nephrol. 11, 1067 (2000). To determine the effect of βpep-25 on physiological parameters, tumor blood perfusion and tumor oxygenation were measured in separately treated groups of animals.

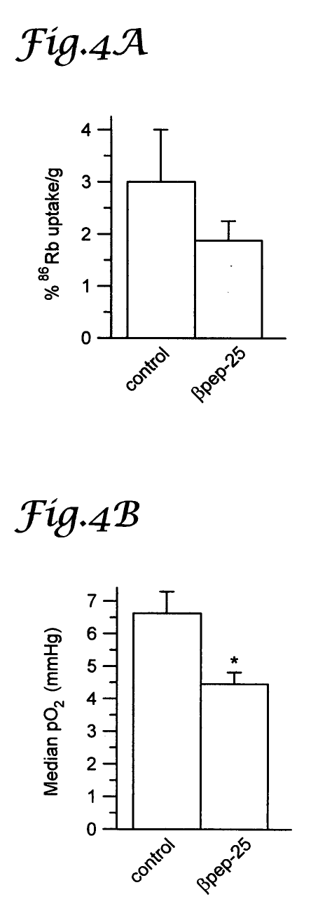

It was found that a limited schedule of βpep-25 treatment (daily injection of 10 mg/kg i.p. for 5 days) of SCK-tumor bearing animals with large tumors (about 300 mm³) resulted in reduced perfusion estimated by ⁸⁶Rb uptake. In control mice, ⁸⁶Rb uptake was 3.0%±1.0% per gram of tumor tissue, and in βpep-25-treated, size-matched tumors this was 1.9%±0.4% per gram of tumor tissue (FIG. 4, p=0.12). For reference, perfusion estimated in major organs (liver, spleen, muscle, kidney and lungs) showed no significant difference between βpep-25-treated and non-treated mice (data not shown). ⁸⁶Rb uptake in SCK tumors was also measured after 14 days of βpep-25 treatment (via osmotic pumps; see tumor growth in FIG. 3). These tumors were relatively large, exceeding an average of 1000 mm³, and no differences in perfusion between βpep-25-treated and control groups were observed (2.85±0.5%/g vs. 2.8±0.4%/g, respectively).

In addition, the median pO₂ was measured in other relatively large SCK tumors from the treatment groups shown in FIG. 4. Five mice were assessed for the control treated group (total measurement points n=199), and six mice in the βpep-25 treated group (total measurement points n=255) resulting in a mean pO₂ of 9.5±1.4 mmHg and 6.0±0.7 mmHg, respectively. After 14 days of continuous, systemic treatment with βpep-25, no difference was found between tumors from βpep-25-treated and control animals. Control tumors had a median pO₂ of 1.4 mmHg, whereas βpep-25-treated animals had a median pO₂ of 1.5 mmHg. Due to the fact that SCK-bearing animals from the tumor response assays used to measure the pO₂ were treated with 20 mg/kg βpep-25, the effect of daily injections of 20 mg/kg βpep-25 on SCK tumor pO₂ was also tested. An independent group of mice bearing SCK tumors (initial tumor size of 50 mm³) treated with 7 daily injections of βpep-25 (20 mg/kg i.p.) had a significantly reduced median tumor pO₂ (p less than 0.05; FIG. 4b). The average median pO₂ in control tumors was 6.7±0.66 mmHg, and this was reduced to 4.5±0.35 mmHg in βpep-25-treated mice with size-matched tumors of about 300 mm³. Within 2 hours after the final injection, blood pressure was taken prior to measuring the pO₂. Blood pressure, which was not affected by 7 daily injections of βpep-25, showed an average systolic pressure of 88.6±12.1 mmHg in control (PBS treated) mice and 85.0±11.8 mmHg in βpep-25-treated mice.

### Example 8

### βpep-25 Functions as a Radiosensitizer In Vivo

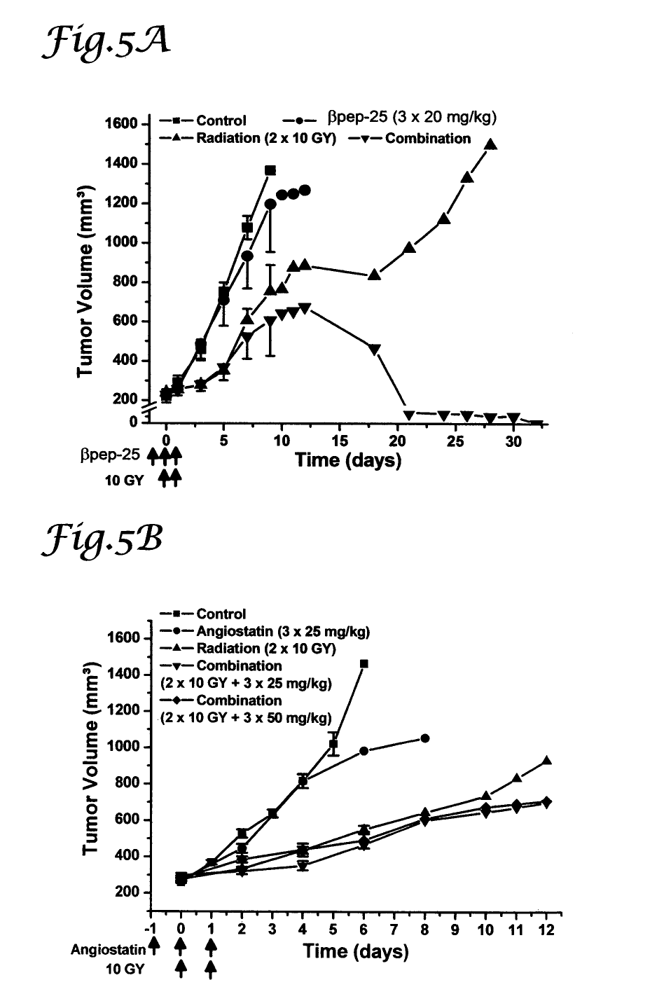

During the course of these combination treatment studies, it was hypothesized that βpep-25 functions by sensitizing endothelial cells to radiation therapy. To test this hypothesis, βpep-25 was administered to SCK tumor-bearing mice via i.p. injection (20 mg/kg on days −1, 0, 1) two hours prior to tumor irradiation (10 Gy locally on days 0 and 1). Compared to tumors from control animals, there was a little effect from βpep-25 alone on tumor growth (FIG. 5a), whereas two radiation treatments (10 Gy each) increased tumor growth delay by approximately 4 days. Combination treatment further delayed tumor growth and resulted in 50% complete responses. Radiation treatment alone produced no complete responses. Based on time to grow 3-fold in volume from initial tumor volume, βpep-25 in combination with radiation resulted in a synergy ratio of 1.6. For comparison, the well-known anti-angiogenic angiostatin (Gorski et al., Cancer Res 58, 5686-9 (1998)) was used with the same treatment regimen (FIG. 5b). For 3 consecutive days (1−, 0, 1), angiostatin (25 or 50 mg/kg) was given i.p. to SCK tumor-bearing mice two hours prior to radiation therapy on days 0 and 1 (10 Gy each). In the fast-growing SCK model, combination of angiostatin and radiation did not result in any significant delay in tumor growth, nor did it induce any complete responses.

### Example 9

### βpep-25 Antitumor Effect with Post-radiation Administration

Experiments were performed using 7 to 8 week old male C3H/HeJ mice. SCCVII tumor cells (squamous cell carcinoma) were grown in vitro using RPMI 1640 medium supplemented with 10% fetal bovine serum and 100 Units (U)/ml penicillin and 0.1 mg/ml streptomycin. Cells in exponential growth phase were harvested by treatment with 0.25% trypsin solution. Approximately 1×10⁶ viable cells were injected subcutaneously (sc) into the right legs of the mice. Radiation treatment was initiated when tumors had grown to approximately 200 mm³ in size (typically 7-10 days after inoculation). Initiation of radiation treatment is defined as day 0, and anginex was administered via i.p. injection on days 1 and 3.

pO₂ in SCCVII tumors was measured using a polarographic electrode (POG-203 Unique Medical Tokyo Japan). Mice were anesthetized with an i.p. injection of 50 mg/kg (i.e., 0.2 ml for a 20 g mouse) sodium pentobarbital (Dainippon Pharmaceutical Co. Ltd., Tokyo, Japan), and anesthetized animals were laid on a Plexiglas board with legs gently stretched and secured by taping the foot to the board. The pO₂ electrode, which was calibrated in 0.9% saline and alternately bubbled with 100% nitrogen and 20.9% oxygen, was inserted by hand to about 1 mm deep into the tumor through a small incision in the skin on the distal side of the tumor. The electrode was then guided by a computer-controlled system through the tumor tissue, and the pO₂ was measured by advancing the electrode 0.7 mm and immediately withdrawing it 0.3 mm to reduce the compression pressure. The pO₂ was measured and recorded by computer. Animals with tumors the size of about 150 mm³ were used for all measurements. The pO₂ was measured 4 days after the first injection of βpep-25 and thus 1 day after the final treatment. Five mice were used for each time point.

A Philips 200-kV orthovoltage machine (Philips Medical Systems, Tokyo, Japan) was used for irradiation both for in vivo and in vitro work. The radiation factors are 200 kVp, 9 mA with an added filtration of 0.2 mm Cu at the final dose rate of 0.419 Gy/min.

For the in vitro work, a set number of cells was added to medium in tissue culture flasks and incubated overnight to allow for cell adherence. The flasks were tightly capped and irradiated with 2.5 Gy or 5 Gy, with or without prior exposure to 10 micromole (μM) of βpep-25 for 2.5, 4, 6, 16 hours. Immediately following exposure to βpep-25, cells were gently rinsed with 4 ml of βpep-25-free medium and cultured with fresh medium under an atmosphere of 95% air and 5% CO₂ for 8 days at 37° C. Colonies were stained with crystal violet and counted. A viable colony was defined as one containing more than 50 cells.

For the in vivo work, tumor sizes averaged 200 mm³ on the day of radiation treatment. Tumor-bearing mice were anesthetized and covered with a 4-mm-thick lead shield. Their legs were gently extended into the X-ray field and exposed to a single dose of 8 Gy. Tumor size was measured using a metric scale caliper every 2 days until the mean tumor volume of each group reached three times the volume on the day of treatment. The tumor volume was calculated using the formula a²b/2, where a and b are the shorter and longer diameters of the tumor, respectively.

An 11 Megahertz (MHz) ultrasound unit (Aplio (SSA 770A); Toshiba Co. Ltd., Tokyo, Japan) was used to examine the effect of βpep-25 on the internal architecture and vascularity of implanted tumors. Power Doppler images were taken to assess flow activity in tumors, as well as in peripheral vessels, in a 150×100×80 mm water bath at 37° C. Images were taken at 7 or 14 days (control and βpep-25 alone: 3 or 7 days) following combination therapy to ensure use of size-matched tumors.

Tumor-bearing mice were treated with 10 mg/kg βpep-25 (2 injections) and/or 8 Gy in a manner identical to the in vivo tumor growth delay studies described above. The investigation of tumor hypoxia was initiated by i.v. injection of 60 mg/kg pimonidazole through the lateral tail vein of tumor-bearing C3H mice. Pimonidazole, a substituted 2-nitroimidazole with a molecular weight of 290.7, is preferentially reduced in hypoxic viable cells and forms irreversible protein adducts, which have been optimized for detection with immunohistochemistry. The plasma half-life of pimonidazole is 0.5 hour in C3H mice. At 3 hour post-injection, the mouse was sacrificed, and the tumor was dissected and immediately fixed in 10% formalin. Following paraffin embedding and sectioning of fixed tumor tissue, a monoclonal antibody against protein adducts of pimonidazole was added. To reveal the location of these adducts, a secondary antibody conjugated with horseradish peroxidase was applied, and images of sections were acquired using a digital CCD camera under brightfield microscopy at 40× and 400× magnification. Statistical analyses were performed using the Student's t-test or Turkey Kramer test. A p-value of 0.05 or less was considered statistically significant.

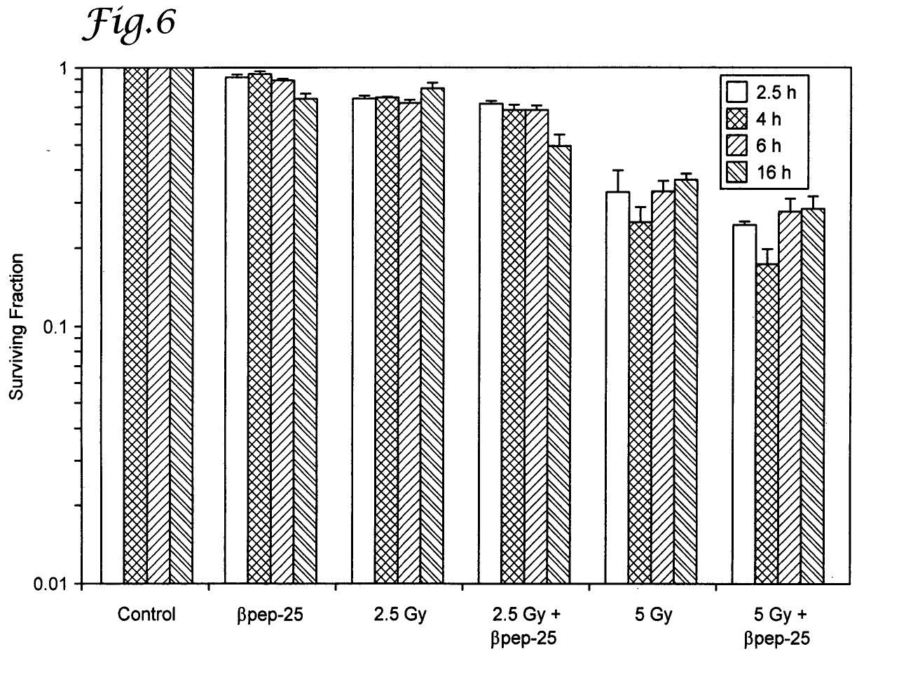

The effect of βpep-25 on radiation-induced tumor growth delay is shown in FIG. 6, which plots the mean (n=7 to 10) relative tumor volume as a function of time after initiation of treatment. The volume of control SCCVII tumors increased three-fold over 6 days, whereas it increased three-fold after 10 days following exposure to 8 Gy irradiation. In mice injected i.p. with 10 mg/kg βpep-25, tumor volume increased three-fold in 7 days, only slightly more than in control animals. However, the delay in tumor growth was further increased to a 10-day delay in animals receiving both radiation and βpep-25 therapy. In other words, tumors receiving combination therapy required 16 days to increase in volume by 3-fold on average, which was found to be significant compared to radiation therapy alone (Student t test, p less than 0.01) and supra-additive.

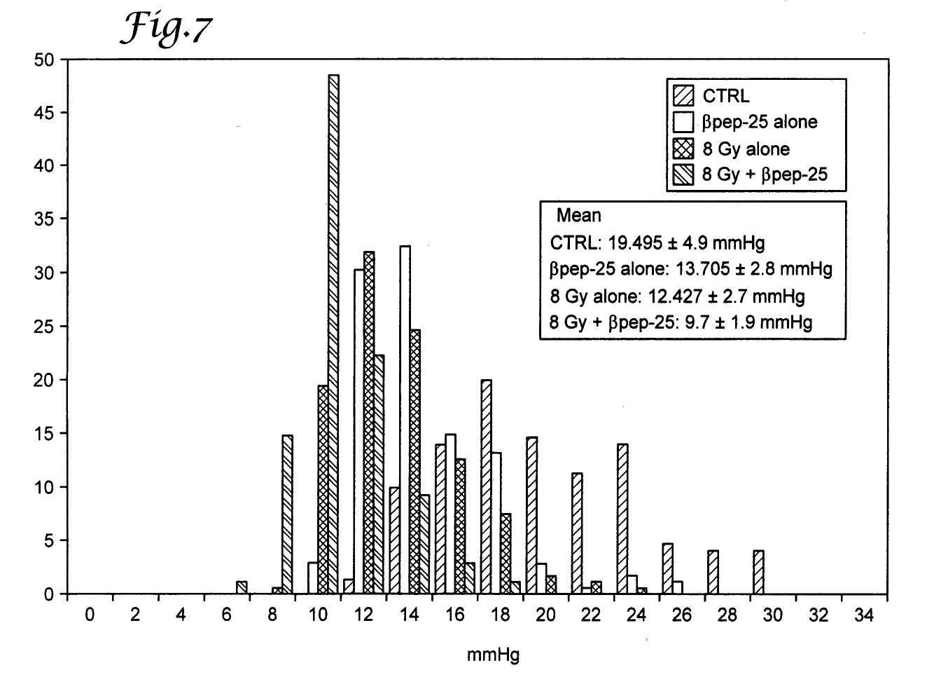

At the end of tumor growth delay experiments, tumors were excised and cross-sections collected and stained as described. Images from microscopic examination of pimonidazole and H&E stained tumors are shown in FIG. 7. Images from control tumors did not show any visible signal, whereas tumors from each treatment group did reveal hypoxic regions as indicated by the brown coloring. Tumors from βpep-25 treated groups generally demonstrated the most hypoxia and evidence of neutrophil invasion.

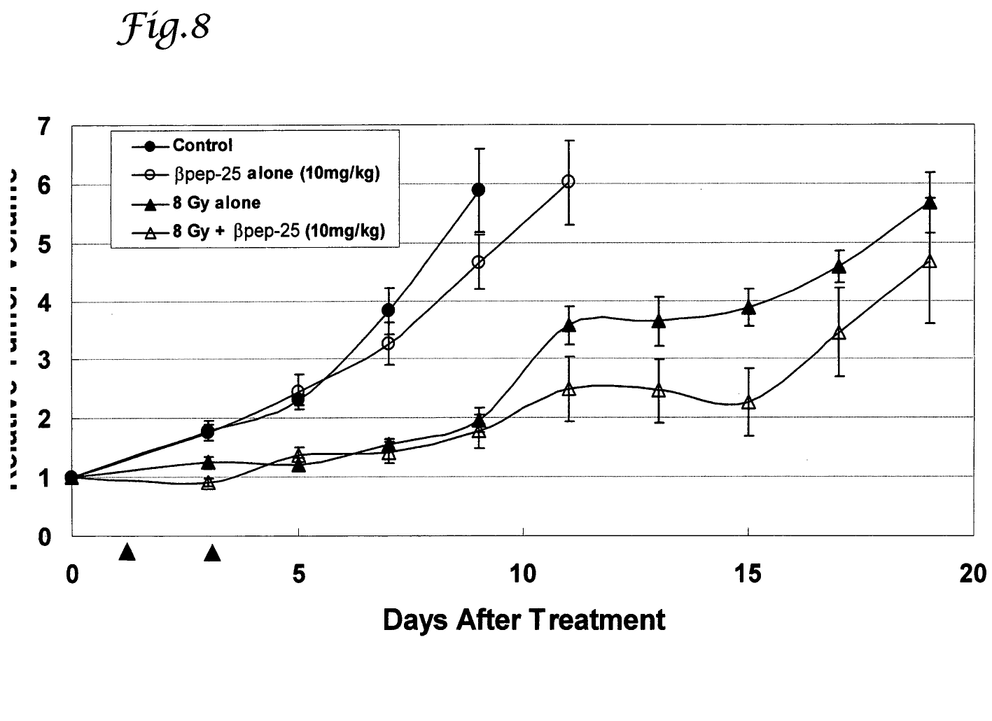

The measurement of pO₂ in SCCVII tumors is shown in FIG. 8, which provides histograms from saline treated control groups, βpep-25 treated groups, 8 Gy irradiated groups, and combination treated groups. These histograms were constructed as a function of oxygen tension collected in 2 mmHg intervals. In saline-treated SCCVII tumors, the mean pO₂ was 19.5±4.9 mmHg (n=100), which decreased to 13.7±2.8 mmHg (n=100) in βpep-25 treated mice and 12.4±2.7 mmHg (n=100) in 8 Gy treated mice. The mean pO₂ was further decreased significantly (p less than 0.01 in a Turkey Kramer test) to 9.7±1.9 mmHg (n=100) in mice treated with a combination of βpep-25 and 8 Gy radiation.

FIG. 9 illustrates power Doppler detected blood flow in SCCVII tumors. This technique allowed differentiation of intra-tumoral vessels and peripheral vessels. Although intra-tumoral and peripheral blood flow was observed to be essentially the same in control and 8 Gy irradiated mice, groups that received βpep-25, either alone or in combination, demonstrated little intra-tumoral blood flow at 14 days after treatment. A lack of toxicity from βpep-25 was evidenced both in vitro and in vivo after exposure to βpep-25 for 2.5, 4, 6 and 16 hours, followed by 2.5 Gy or 5 Gy radiation.

### Example 10

### βpep-25 Selectively Radiosensitizes Endothelial Cells In Vitro

Human umbilical vein derived EC (HUVEC) and human microvascular EC (MEC) were harvested and cultured as described by Griffioen et al. (Biochem J. 354, 233-42 (2001)). MA148, a human epithelial ovarian carcinoma cell line, and SCK, a mammary carcinoma cell line was cultured as described by Yokoyama et al. (Cancer Res. 60, 2190 (2000)).

For the proliferation assay, HUVEC were seeded in a 96-well culture plate coated with 0.2% gelatin (2 hours at 20° C.). MA148 cells and SCK cells were seeded in non-coated 96-well plates. All cell types were seeded at a concentration of 3,000 cells per well and allowed to adhere for at least 3 hours at 37° C. in 5% CO₂/95% air before treatments were initiated. The cells were then exposed to complete medium with 20 ng/ml basic Fibroblast Growth Factor (bFGF), with or without various concentrations of βpep-25 for 72 hours or as indicated otherwise. In the groups receiving radiation, βpep-25 was added to the wells 4 hours prior to radiation exposure and the plates were then returned to the incubator until 72 hours had elapsed. [³H]-thymidine incorporation or the cell counting kit (CCK-8) were used to assess cell proliferation rates relative to untreated cells, as described by Dings et al. (Cancer Lett. 194, 55 (2003). All measurements were done in triplicate, and the experiments were done at least three times.

An in vitro cell clonogenic assay was conducted according to the following procedure. Cells in exponential growth phase were trypsinized, washed, counted and seeded into 25 cm² tissue culture flasks in duplicate for all conditions. The flasks were incubated overnight at 37° C., exposed to βpep-25 (2 μM) for 4 hours and then irradiated, rinsed once with normal medium, and incubated with fresh medium for 7-10 days in a 5% CO₂/95% air, 37° C. incubator. The resulting colonies were stained with crystal violet in methanol/acetic acid (10:1) and counted by hand, as described by Ahn et al. (Int. J. Radiat. Oncol. Biol. Phys. 57, 813 (2003)).

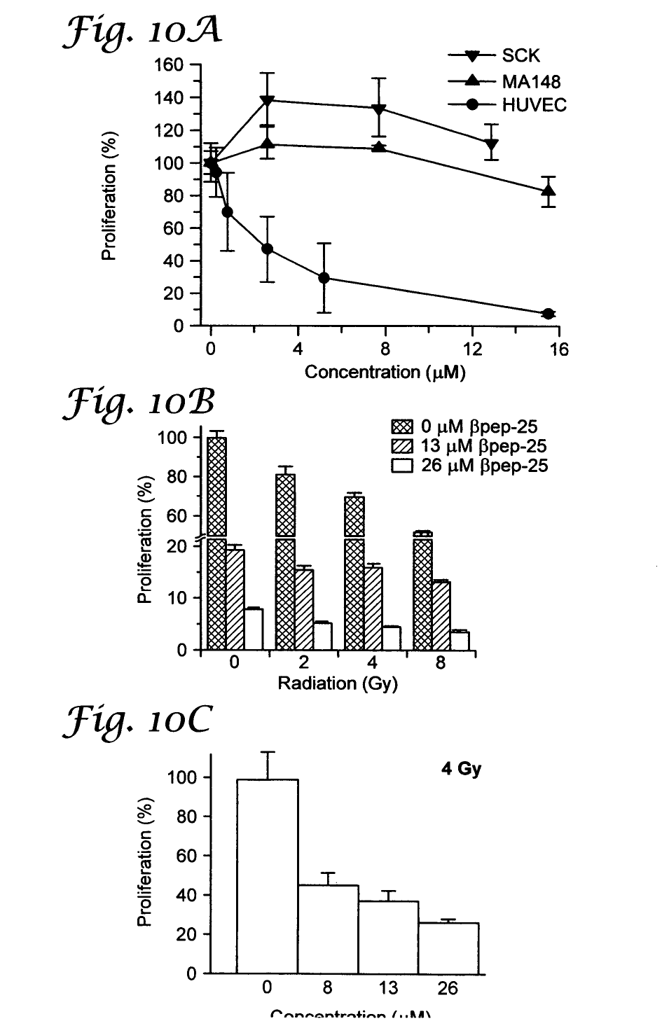

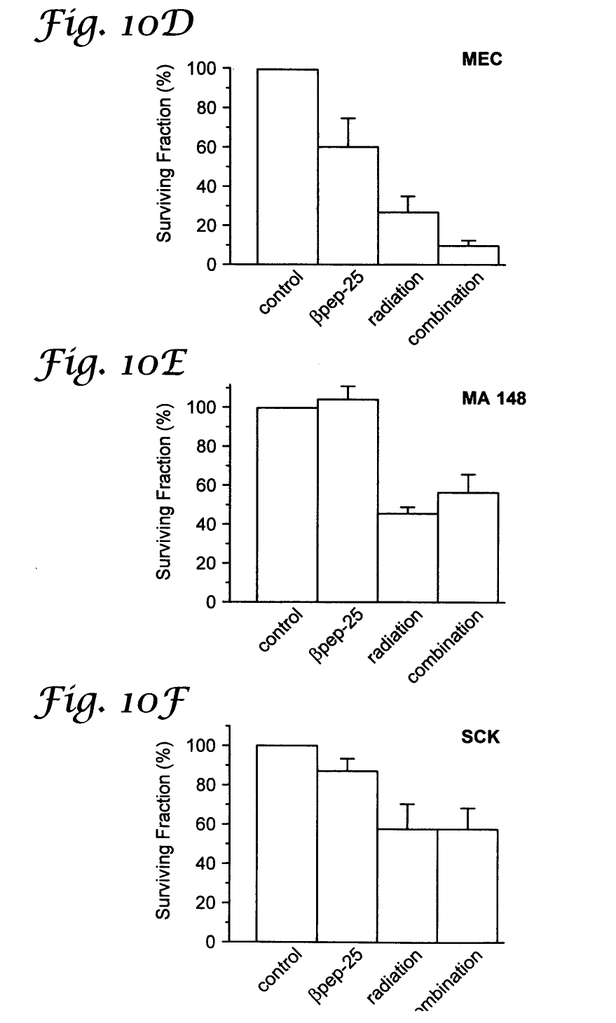

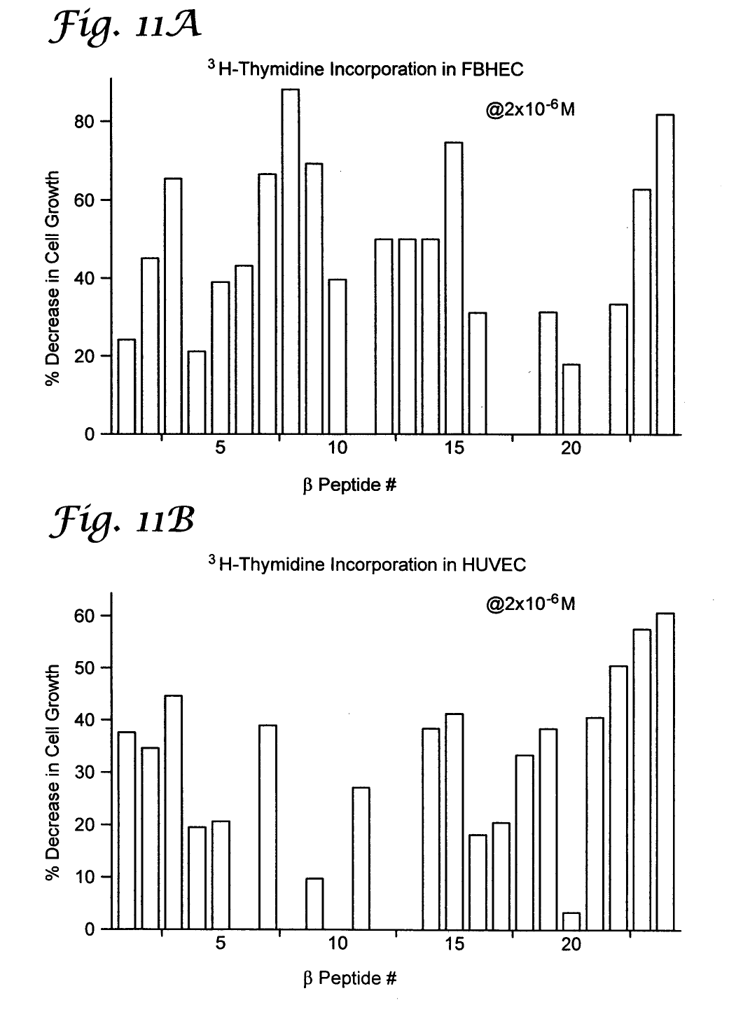

To further demonstrate that βpep-25 functions as a radiosensitizer, a series of in vitro cell culture experiments (proliferation and colony formation assays) were performed using HUVEC, MEC, MA148 and SCK cells. Both assays produced comparable results. In the proliferation assay, βpep-25 alone for 72 hours inhibited endothelial cell proliferation dose dependently, but had no effect on MA148 or SCK cell growth (FIG. 11a). Treatment with βpep-25, 4 hours before exposure to 0-8 Gy, markedly decreased HUVEC growth when measured at 72 hours (FIG. 10a) compared to radiation treatment alone. In a related study, HUVEC were exposed to βpep-25 for only 24 hours before administering a single dose of radiation (4 Gy). Prior to irradiation, βpep-25 was washed away, and HUVEC were provided fresh cell culture media. EC viability was assessed 72 hours after the start of the experiment (48 hours after radiation) by measuring the extent of cell proliferation. In a concentration dependent manner, βpep-25 again enhanced the effect of radiation on EC (FIG. 10c).

In the colony-formation assay, a 4 hour exposure to 2 μM βpep-25 (IC₅₀ for HUVEC proliferation) was used in combination with a sub-optimal dose 2.5 or 5 Gy of radiation. βpep-25 had no effect on either tumor cell line, therefore the IC₅₀ of βpep-25 against HUVEC and MEC cells was used for tumor cells (2 JM). βpep-25 was then removed following irradiation and cells were cultured in fresh medium. For this assay, MEC primary cultures were used as a model of microvascular cells and because HUVECs do not reliably form colonies. While 4 hours of βpep-25 or 2.5 Gy alone reduced MEC clonogenicity on average by 40% and 76%, respectively, 4 hours of βpep-25 exposure followed by 2.5 Gy reduced clonogenicity by 91% (FIG. 10d). In contrast, survival of MA148 and SCK tumor cells treated with 5 Gy reduced cell survival by about 50%. βpep-25 exposure for 4 hours alone did not significantly reduce tumor cell survival, and treatment with βpep-25 for 4 hours followed by irradiation with 5 Gy did not reduce survival compared to radiation alone (FIGS. 10e and 10f).

### Example 11

### Angiogenesis Inhibition by β-sheet Peptides

Platelet Factor 4 (PF4), one of the most potent angiogenesis inhibitors, and related βpep-peptides were tested for their effects on endothelial adhesion molecule expression from endothelial cells (EC). Like native PF4, βpep-14 and βpep-16 were found to be angiostatic as determined by measurement of EC proliferation in vitro (Table 3). The effect of the peptides on expression of intercellular adhesion molecule-1 (ICAM-1) was also tested.

In a first series of experiments, PF4 and βpep peptides were tested for their ability to prevent bFGF (fibroblast growth factor) mediated downregulation of ICAM-1. It was found that inhibition of angiogenesis and endothelial cell proliferation resulted in a complete blockade of bFGF mediated ICAM-1 downregulation. A 3-day preincubation of EC with 10 ng/ml bFGF resulted in a marked modulation of ICAM-1. Simultaneously 100 μg/ml of each PF4, βpep-14, βpep-16, or medium was added. Mean ICAM-1 fluorescence intensity values were determined. The addition of 100 μg/ml PF4 enhanced the expression of ICAM-1. Simultaneous addition of bFGF and PF4 did not result in the loss of ICAM-1 expression. Also, the addition of the PF4 related peptides βpep-14 and βpep-16 resulted in a complete block of bFGF-mediated downregulation.

Since the in vivo situation of tumor-associated EC involves the low expression or even absence of ICAM-1, the next set of experiments aimed to study the ability to re-induce ICAM-1 expression after bFBF preincubation. It had been demonstrated previously that the longevity of the bFGF mediated ICAM-1 downregulation is at least 7 days. Treatment of EC expressing downregulated ICAM-1 levels with 100 μg/ml PF4 resulted, even in the presence of bFGF, in reinduction of ICAM-1. βpep-14 and βpep-16 showed similar results. In these experiments, HUVEC were pretreated for 3 days with bFGF, subsequently PF4 was added for 3 days and, where indicated in the last 16 hours of culture 4 ng/ml TNF(X was added. Human umbilical vein derived endothelial cells (HUVEC) were harvested from normal human umbilical cords by perfusion with 0.125% trypsin/EDTA. Cells were cultured in fibronectin (FN) coated tissue culture flasks in culture medium (RPMI-1640 with 20% human serum (HS), supplemented with 2 mM glutamine and 100 U/ml penicillin and 0.1 mg/ml streptomycin). Immunofluorescence using indirect PE-conjugated reagents required three separate incubations. 1×10⁵ EC were fixed for 1 hour in 1% paraformaldehyde, resuspended in 20 μl appropriately diluted Mab and incubated for 1 hour on ice. Subsequently, cells were washed two times in PBS/BSA (0.1%) and incubated for another 30 minutes with biotinylated rabbit-anti-mouse Ig (Dako, Glostrup, Denmark). After another 2 washings, cells were incubated with streptavidin-phycoerythrin conjugate (Dako). Stained cells were analyzed on a FACScan flowcytometer. Data analysis was performed using PCLysys software (Becton Dickinson, Mountain View, Calif.). Statistical significance of observed differences was determined using the Student's t-test.

The anergy of EC to stimulation with inflammatory cytokines was the subject of additional experiments. For these experiments, HUVEC (human vascular endothelial cells) were pretreated with 10 ng/ml bFGF for 3 days. Subsequently, cells are subcultured for 3 days with 100 μg/ml bFGF in the presence of PF4. For the last 16 hours of the culture 4 ng/ml TNF-α was added to induce upregulation of ICAM-1. The decreased inflammatory response of angiogenic stimulated EC was found to be overcome by simultaneous treatment with PF4 and similar results were found for βpep-14 and βpep-16. The regulation of ICAM-1 at the protein level was confirmed in Northern blot analysis for detection of ICAM-1 message. In these experiments, HUVEC were cultured for 3 days with bFGF and treated for the last 4 and 24 hours with PF4 (100 μg/ml). TNF-α was added 2 hours before isolation of RNA. RNA from a subconfluent EC cultures (75 cm² Petri-dishes) incubated with bFGF for different time-points was isolated using an RNA-zol kit (Campro Scientific, Houston, Tex.). Total RNA (10 μg) for each sample was separated in a 0.8% formaldehyde-denaturing gel, transferred to nitrocellulose (Hybond N+, Amersham International, Amersham, UK) and hybridized to a ³²P-labelled 1.9 Kb c-DNA probe, containing the functional sequence of the human ICAM-1 gene (a gift from Dr. B. Seed). Membranes were washed at a high stringency in 0.2×SSC, 0.1% SDS at 50° C. for 1 hour. Filters were exposed to X-ray films (Kodak X-omat, Eastman Kodak Company, Rochester, N.Y.) using an intensifying screen at −80° C. for not less than 12 hours. Autoradiograms were subjected to scanning using a laser densitometer (Model GS670, Bio-Rad, Hercules, Calif.) and data were analysed with the Molecular Analyst PCTM software. The intensity of the major ICAM-1 mRNA transcript was normalized with respect to actin mRNA expression used as a control.

Functional impact for the observed phenomena was provided by leukocyte/EC adhesion assays as described earlier (Griffloen et al. Cancer Res. 56, 1111-1117 (1996)). The bFGF mediated inhibition of leukocyte adhesion to cultured HUVEC was completely abolished in the presence of PF4 or related peptides. TNF mediated upregulation of adhesion to bFGF preincubated HUVEC in the presence of PF4 was similar to the adhesion to TNF treated control cells. PHA-activated peripheral blood T lymphocyte were adhered for 1 hour at 37° C. to TNF-α (4 ng/ml), bFGF (10 ng/ml) and PF4 (100 μg/ml) treated, or control (HUVEC). Non-adhering cells were removed and adhered cells were enumerated by an inverted microscope. Values of one representative experiment out of three are expressed as numbers of adhered cells per high power field. Statistical significance is determined by the Student's t-test.

These results indicate that the inhibition of angiogenesis and endothelial cell proliferation, which has been demonstrated to prevent outgrowth of solid tumors and metasteses, is able to overcome the downregulation of adhesion molecules and the anergy upon stimulation with inflammatory cytokines. In experiments to document the effect of other inhibitors of angiogenesis the same results were found for thrombospondin-1 and IP-10. However, the metalloproteinase inhibitor BB-94 (batimastat) and thalidomide, which do not affect EC growth in vitro, did not affect ICAM-1 expression. It can thus be concluded that the ICAM-1 regulation coincides with the regulatory mechanisms involving EC growth. The present results indicate that adhesion molecules which are necessary for the formation of an efficient leukocyte infiltrate are not only under regulation of angiogenic factors but are induced under conditions of angiogenesis inhibition.

TABLE 3
INHIBITION OF EC-PROLIFERATION (³H)-THYMIDINE
INCORPORATION BY DIFERENT ANTIOGENESIS INHIBITORS

| | no bFGF | 10 ng/ml bFGF |
|---|---|---|
| expt 1 | | |
| medium | 4044 ± 206 | 28815 ± 1007 |
| PF4 (1 μg/ml) | 4656 ± 456 | 28782 ± 815 |
| PF4 (10 μg/ml) | 4066 ± 351 | 23868 ± 402 |
| PF4 (100 μg/ml) | 1651 ± 172 | 4655 ± 421 |
| expt 2 | | |
| medium | 14296 ± 2490 | 29079 ± 2506 |
| βpep-14 (1 μg/ml) | 14184 ± 1775 | 28695 ± 1062 |
| βpep-14 (10 μg/ml) | 9886 ± 2114 | 29530 ± 1608 |
| βpep-14 (100 μg/ml) | 3774 ± 299 | 6585 ± 132 |
| βpep-16 (1 μg/ml) | 15039 ± 2020 | 35447 ± 2621 |
| βpep-16 (10 μg/ml) | 11881 ± 2545 | 33663 ± 2572 |
| βpep-16 (100 μg/ml) | 4929 ± 749 | 7852 ± 875 |
| expt 2 | | |
| medium | 6780 ± 713 | 52808 ± 4092 |
| PF4 (1 μg/ml) | 6171 ± 1227 | 43524 ± 5318 |
| PF4 (10 μg/ml) | 3547 ± 317 | 8337 ± 704 |
| PF4 (100 μg/ml) | 947 ± 170 | 1654 ± 375 |
| βpep-14 (1 μg/ml) | 7214 ± 1668 | 48443 ± 2700 |
| βpep-14 (10 μg/ml) | 6074 ± 899 | 52126 ± 1258 |
| βpep-14 (100 μg/ml) | 1062 ± 325 | 7663 ± 715 |
| βpep-16 (1 μg/ml) | 7450 ± 737 | 47727 ± 447 |
| βpep-16 (10 μg/ml) | 6148 ± 1370 | 44919 ± 2081 |
| βpep-16 (100 μg/ml) | 2669 ± 370 | 27071 ± 3277 |
| expt 3 | | |
| medium | ND | 3432 ± 232 |
| IP-10 (100 μg/ml) | ND | 725 ± 95 |
| expt 4 | | |
| medium | 18904 ± 1501 | 31954 ± 1220 |
| TSP-1 (10 μg/ml) | 8865 ± 639 | 22338 ± 860 |
| TSP-1 (25 μg/ml) | 5565 ± 349 | 10267 ± 797 |

EC proliferation was measured using a ³[H]thymidine incorporation assay. EC were seeded in flatbottomed 96-well tissue culture plates (5000 cells/well) and grown for 3 days, in culture medium. In some cultures the proliferation of EC was enhanced by incubation with 10 ng/ml bFGF. During the last 6 hours of the assay, the culture was pulsed with 0.5 μCi [methyl-3 H]thymidine/well. Results are expressed as the arithmetic mean counts per minute (cpm) of triplicate cultures.

β-sheet peptides βpep 1-24 were tested in an endothelial cell proliferation assay using ³H-thymidine incorporation. At least half of the peptides were somewhat active at 2.6 micromolar at decreasing endothelial cell growth. These results are provided in FIG. 7. βpep-23 and βpep-24 were about 30% effective at 0.26 μM.

The peptides were also able to regulate inter-cellular adhesion molecule (ICAM) expression. This receptor is downregulated in tumors and agents that are effective at upregulating ICAM are potentially useful therapeutic agents to control tumor growth. Those that were the most anti-angiogenic appeared to be least effective at ICAM regulation. That the β-sheet peptides have the same or similar positive charge to mass ratios but do not share the same activities indicates that the peptides work specifically. For example, βpep-8 deomonstrates little cell proliferation activity while βpep-24 was very good at controlling cell proliferation. Those skilled in the art will readily be able to use the assays provided here and the βpep sequences disclosed herein to identify peptides with ICAM upregulating activity and peptides with endothelial cell proliferation activity without undue experimentation.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method of treating a patient with a tumor, the method comprising:
delivering radiation to the patient; and
administering to the patient a β-sheet peptide comprising a water soluble peptide having at least 35% to 55% amino acids having hydrophobic side chains,
wherein the ratio of amino acids having positively charged side chains amino acids to amino acids having negatively charged side chains is at least 2: 1,
wherein at least two of the amino acids having hydrophobic side chains are positioned in
the peptide chain with an intervening turn sequence in a manner such that the two amino acids having hydrophobic side chains are capable of aligning in a pairwise fashion to form a β-sheet structure,
wherein the peptide is water soluble under physiological conditions, and
wherein the peptide forms β-sheet structures.

2. The method of claim 1, wherein the turn sequence is LXXGR (SEQ ID NO:33) and X is independently selected from the group consisting of K, N, S, and D.

3. The method of claim 1, wherein the β-sheet peptide consists of 28 to 33 amino acids.

4. The method of claim 2, wherein the β-sheet peptide consists of about 28 to 33 amino acids.

5. The method of claim 1, wherein the radiation and the β-sheet peptide treat the patient with a tumor synergistically.

6. The method of claim 1, wherein the β-sheet peptide is administered before delivering radiation to the patient and wherein the β-sheet peptide radiosensitizes the tumor to radiation.

7. The method of claim 6, wherein the β-sheet peptide radiosensitizes endothelial cells.

8. The method of claim 1, wherein the β-sheet peptide is administered within 24 hours of delivering radiation to the patient.

9. The method of claim 1, wherein the β-sheet peptide is delivered in a pharmaceutically acceptable carrier.

10. The method of claim 1, wherein the method includes inhibition of angiogenesis by the β-sheet peptide.

11. The method of claim 1, wherein the radiation comprises gamma ray or x-ray radiation.

12. The method of claim 1, wherein the tumor is a solid tumor.

13. The method of claim 12, wherein the solid tumor is selected from the group consisting of carcinomas, sarcomas, and lymphomas.

14. The method of claim 12, wherein the radiation comprises a daily dose of about 50 to 70 grays.

15. The method of claim 12, wherein the solid tumor is present in the brain, breast, cervix, larynx, lung, pancreas, prostate, skin, spine, stomach, or uterus.

16. The method of claim 1, wherein the tumor is a leukemia.

17. The method of claim 16, wherein the radiation comprises a daily dose of about 20 to 40 grays.

18. The method of claim 1, wherein the β-sheet peptide is selected from the group consisting of βpep-1 through βpep-30 (SEQ ID NOS:1-30) and their derivatives.

19. The method of claim 1, wherein the β-sheet peptide is βpep-25 and its derivatives.

20. The method of claim 1, wherein the β-sheet peptide is βpep-25.

21. A method of treating a patient with a tumor, comprising:
delivering gamma or x-ray radiation to the patient; and
administering to the patient βpep-25 in a pharmaceutically acceptable carrier.

22. The method of claim 21, wherein the radiation and βpep-25 treat the patient with a tumor synergistically.

23. The method of claim 22, wherein the radiation and βpep-25 provide a synergistic effect of 200% or more.


SEQUENCE LISTING (34 sequences)

SEQ ID NO: 1 - protein, 33 aa, artificial sequence
  chemically synthesized
KFIVTLRVIKAGPHSPTAQIIVELKNGRKLSLD

SEQ ID NO: 2 - protein, 33 aa, artificial sequence
  chemically synthesized
ANIKLSVEMKLFKRHLKWKIIVKLNDGRELSLD

SEQ ID NO: 3 - protein, 33 aa, artificial sequence
  chemically synthesized
  misc_feature: d-isomer of tryptophan
  misc_feature (15)..(15): Xaa can be any naturally occurring amino acid
ANIKLSVEMKLFCYXKVCKIIVKLNDGRELSLD

SEQ ID NO: 4 - protein, 33 aa, artificial sequence
  chemically synthesized
SIQDLNVSMKLFRKQAKWKIIVKLNDGRELSLD

SEQ ID NO: 5 - protein, 33 aa, artificial sequence
  chemically synthesized
ANIKLSVKWKAQKRFLKMSINVDLSDGRELSLD

SEQ ID NO: 6 - protein, 33 aa, artificial sequence
  chemically synthesized
HIKELQVKWKAQKRFLKMSIIVKLNDGRELSLD

SEQ ID NO: 7 - protein, 33 aa, artificial sequence
  chemically synthesized
SIQDLNVSMKLFRKQAKWKINVKLNDGRELSLD

SEQ ID NO: 8 - protein, 33 aa, artificial sequence
  chemically synthesized
HIKELQVRWRAQKRFLRMSIIVKLNDGRELSLD

SEQ ID NO: 9 - protein, 33 aa, artificial sequence
  chemically synthesized
HIKELQVKMKAQKRFLKWSIIVKLNDGRELSLD

SEQ ID NO: 10 - protein, 33 aa, artificial sequence
  chemically synthesized
ANIKLSVKWKAQKRFLKMSIIVKLNDGRELSLD

SEQ ID NO: 11 - protein, 33 aa, artificial sequence
  chemically synthesized
ANIKLSVEMKLFCRHLKCKIIVKLNDGRELSLD

SEQ ID NO: 12 - protein, 33 aa, artificial sequence
  chemically synthesized
ANIKLSVEMKFFKRHLKWKIIVKLNDGRELSLD

SEQ ID NO: 13 - protein, 33 aa, artificial sequence
  chemically synthesized
ANIKLSVEFKLFKRHLKWKIIFKLNDGREFSLD

SEQ ID NO: 14 - protein, 33 aa, artificial sequence
  chemically synthesized
SIQDLNVSMKLFRKQAKWKLIVKLNDGRELSLD

SEQ ID NO: 15 - protein, 33 aa, artificial sequence
  chemically synthesized
SIQDLNVSMKLFRKQAKWKIILKLNDGRELSLD

SEQ ID NO: 16 - protein, 33 aa, artificial sequence
  chemically synthesized
SIQDLNVSMKLFRKQAKWKIIAKLNDGRELSLD

SEQ ID NO: 17 - protein, 33 aa, artificial sequence
  chemically synthesized
SIQDLNVSMKLFRKQAKWKILVKLNDGRELSLD

SEQ ID NO: 18 - protein, 33 aa, artificial sequence
  chemically synthesized
SIQDLKVSMKLFRKQAKWKIIVKLNDGRELSLD

SEQ ID NO: 19 - protein, 33 aa, artificial sequence
  chemically synthesized
SIQKLNVSMKLFRKQAKWKIIVKLNDGRELSLD

SEQ ID NO: 20 - protein, 33 aa, artificial sequence
  chemically synthesized
  misc_feature: non-common aminoacid norleucine
  misc_feature (10)..(10): Xaa can be any naturally occurring amino acid
SIQDLNVSMXLFRKQAKWKIIVKLNDGRELSLD

SEQ ID NO: 21 - protein, 33 aa, artificial sequence
  chemically synthesized
SIQDLNVSLKLFRKQAKWKIIVKLNDGRELSLD

SEQ ID NO: 22 - protein, 33 aa, artificial sequence
  chemically synthesized
SIQDLNLSMKLFRKQAKWKIIVKLNDGRELSLD

SEQ ID NO: 23 - protein, 33 aa, artificial sequence
  chemically synthesized
SIQDLKVSLNLFRKQAKWKIIVKLNDGRELSLD

SEQ ID NO: 24 - protein, 33 aa, artificial sequence
  chemically synthesized
SIQFLKVSLNLDRKQAKWKIIVKLNDGRELSLD

SEQ ID NO: 25 - protein, 33 aa, artificial sequence
  chemically synthesized
ANIKLSVQMKLFKRHLKWKIIVKLNDGRELSLD

SEQ ID NO: 26 - protein, 33 aa, artificial sequence
  chemically synthesized
SIQDLNVSMKLFRKQAKWKIIIKLNDGRELSLD

SEQ ID NO: 27 - protein, 33 aa, artificial sequence
  chemically synthesized
SIQDLNVSMKLFRKQAKWKAIVKLNDGRELSLD

SEQ ID NO: 28 - protein, 33 aa, artificial sequence
  chemically synthesized
SIQDLNVSMKLFRKQAKWKVIVKLNDGRELSLD

SEQ ID NO: 29 - protein, 33 aa, artificial sequence
  chemically synthesized
SIQDLNVSMKLFRKQAKWKLILKLNDGRELSLD

SEQ ID NO: 30 - protein, 33 aa, artificial sequence
  chemically synthesized
SIQDLNVSMKLFRKQAKWKVIIKLNDGRELSLD

SEQ ID NO: 31 - protein, 33 aa, artificial sequence
  chemokine
RHITSLEVIKAGPHSPTAQLIATLKNGRKISLD

SEQ ID NO: 32 - protein, 33 aa, artificial sequence
  chemokine
KFIKELRVIESGPHSANTEIIVKLSDGRELSLD

SEQ ID NO: 33 - protein, 5 aa, artificial sequence
  turn sequence
  misc_feature: xaa can be any naturally occurring amino acid
  misc_feature (2)..(3): Xaa can be any naturally occurring amino acid
LXXGR

SEQ ID NO: 34 - protein, 33 aa, artificial sequence
  chemokine
KNIQSVNVKSPGPHSAQTEVIATLKNGRKASLN